(19) United States
(12) Patent Application Publication
(10) Pub. No.: US 2014/0012032 A1
(43) Pub. Date: Jan. 9, 2014

(54) METHOD FOR PRODUCTION OF F-18 LABELED AMYLOID BETA LIGANDS

(71) Applicants: Mathias Berndt, Berlin (DE); Lutz Lehmann, Berlin (DE); Uwe Ackermann, Melbourne (AU)

(72) Inventors: Mathias Berndt, Berlin (DE); Lutz Lehmann, Berlin (DE); Uwe Ackermann, Melbourne (AU)

(73) Assignee: PIRAMAL IMAGING SA, Matran (CH)

(21) Appl. No.: 13/702,002

(22) PCT Filed: May 30, 2011
(86) PCT No.: PCT/EP2011/058817

§ 371 Date: Jul. 12, 2013

(30) Foreign Application Priority Data
Jun. 4, 2010 (EP) 10164946.5

(51) Int. Cl. C07C 213/08; C07C 309/73

(52) U.S. Cl. CPC C07C 213/08; C07C 309/73
USPC 558/51; 564/412

(57) ABSTRACT

This invention relates to methods, which provide access to F-18 labeled stilbene derivatives.

### FIELD OF INVENTION

This invention relates to compounds and methods, which provide access to F-18 labeled stilbene derivatives.

### BACKGROUND

4-[(E)-2-(4-{2-[2-(2-fluoroethoxy)ethoxy]ethoxy}phenyl)vinyl]-N-methylaniline has been labeled with [F-18]fluoride and is claimed by patent application WO2006066104 and members of the corresponding patent family.

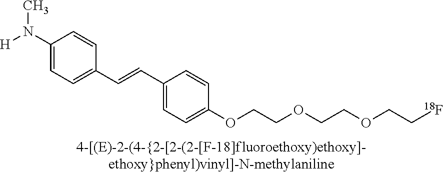 [SMILES: CNc1ccc(/C=C/c2ccc(OCCOCCOCC[18F])cc2)cc1]

The usefulness of this radiotracer for the detection of Aβ plaques have been reported in the literature (W. Zhang et al., Nuclear Medicine and Biology 32 (2005) 799-809; C. Rowe et al., Lancet Neurology 7 (2008) 1-7).

The synthesis of 4-[(E)-2-(4-{2-[2-(2-[F-18]fluoroethoxy)ethoxy]ethoxy}phenyl)-vinyl]-N-methylaniline has been described before:
a) W. Zhang et al., Nuclear Medicine and Biology 32 (2005) 799-809.

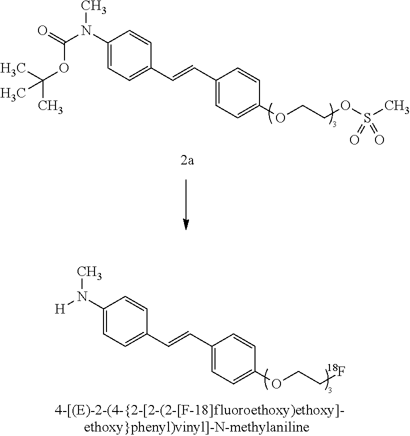

4 mg precursor 2a (2-[2-(2-{4-[(E)-2-{4-[(tert-butoxycarbonyl)(methyl)amino]-phenyl}vinyl]phenoxy}ethoxy)ethoxy]ethyl methanesulfonate) in 0.2 mL DMSO were reacted with [F-18]fluoride/kryptofix/potassium carbonate complex. The intermediate was deprotected with HCl and neutralized with NaOH. The mixture was extracted with ethyl acetate. The solvent was dried and evaporated, the residue was dissolved in acetonitrile and purified by semi-preparative HPLC. 20% (decay corrected), 11% (not corrected for decay) 4-[(E)-2-(4-{(2-[2-(2-[F-18]fluoroethoxy)ethoxy]ethoxy}phenyl)vinyl]-N-methylaniline were obtained in 90 min.
b) WO2006066104
   4 mg precursor 2-[2-(2-{4-[(E)-2-{4-[(tert-butoxycarbonyl)(methyl)amino]-phenyl}vinyl]phenoxy}ethoxy)ethoxy]ethyl methanesulfonate in 0.2 mL DMSO were reacted with [F-18]fluoride/kryptofix/potassium carbonate complex. The intermediates was deprotected with HCl and neutralized with NaOH. The mixture was extracted with ethyl acetate. The solvent was dried and evaporated, the residue was dissolved in acetonitrile and purified by semi-preparative HPLC. 30% (decay corrected), 17% (not corrected for decay) 4-[(E)-2-(4-{(2-[2-(2-[F-18]fluoroethoxy)ethoxy]ethoxy}phenyl)vinyl]-N-methylaniline were obtained in 90 min.
c) WO2010000409
   4 mg of a perfluorinated precursor were reacted with 1.3 GBq [F-18]fluoride to yield N-Boc protected 4-[(E)-2-(4-{2-[2-(2-[F-18]fluoroethoxy)ethoxy]ethoxy}phenyl)vinyl]-N-methylaniline. The unreacted perfluorinated precursor was removed using a fluorous phase cartridge. Deprotection, final purification and formulation to obtain a product, suitable for injection into human is not disclosed. Furthermore, the usefulness (e.g. regarding unwanted F-19/F-18 exchange) of this approach at a higher radioactivity level is not demonstrated. Finally, this method would demand a two-pot setup (first reaction vessel: fluorination, followed by solid-phase-extraction, and deprotection in the second reaction vessel).
   However, the focus of the present invention are compounds and methods for an improved “one-pot process” for the manufacturing of 4-[(E)-2-(4-{2-[2-(2-[F-18]fluoroethoxy)ethoxy]ethoxy}phenyl)vinyl]-N-methylaniline.

Very recently, further methods have been described:
d) US20100113763
   The mesylate precursor 2a was reacted with [F-18]fluoride species in a solvent mixture consisting of 100 μL acetonitrile and 500 μL tertiary alcohol. After fluorination for 10 min at 100-150° C., the solvent was evaporated. After deprotection (1 N HCl, 5 min, 100-120° C.), the crude product was purified by HPLC (C18 silica, acetonitrile/0.1M ammonium formate).
e) H. Wang et al., Nuclear Medicine and Biology 38 (2011) 121-127
   5 mg precursor 2a (2-[2-(2-{4-[(E)-2-{4-[(tert-butoxycarbonyl)(methyl)amino]-phenyl}vinyl]phenoxy}ethoxy)ethoxy]ethyl methanesulfonate) in 0.5 mL DMSO were reacted with [F-18]fluoride/kryptofix/potassium carbonate complex. The intermediate was deprotected with HCl and neutralized with NaOH. The crude product was diluted with acetonitrile/0.1M ammonium dformate (6/4) and purified by semi-preparative HPLC. The product fraction was collected, diluted with water, passed through a C18 cartridge and eluted with ethanol, yielding 17% (not corrected for decay) 4-[(E)-2-(4-{2-[2-(2-[F-18]fluoroethoxy)ethoxy]ethoxy}phenyl)vinyl]-N-methylaniline within 50 min.
   In the paper, the conversion of an unprotected mesylate precursor (is described:
   5 mg unprotected mesylate precursor (2-{2-[2-(4-{(E)-2-[4-(methylamino)phenyl]vinyl}phenoxy)ethoxy]-ethoxy}ethyl 4-methanesulfonate) in 0.5 mL DMSO were reacted with [F-18]fluoride/kryptofix/potassium carbonate complex. The crude product was diluted with acetonitrile/0.1 M ammonium formate (6/4) and purified by semi-preparative HPLC. The product fraction was collected, diluted with water, passed through a C18 cartridge and eluted with ethanol, yielding 23% (not corrected for decay) 4-[(E)-2-(4-{2-[2-(2-[F-18]fluoroethoxy)ethoxy]ethoxy}phenyl)vinyl]-N-methylaniline within 30 min. Beside the purification by HPLC, a process based on solid-phase-extraction was investigated, but the purity was inferior to that with HPLC purification.

So far, one-pot radiolabelings have been performed using a mesylate precursor. It is know, that for F-18 labeling of stilbenes, mesylates have advantages over corresponding tosylates by providing more clean reactions with less amount of by-products (W. Zhang et al. Journal of Medicinal Chemistry 48 (2005) 5980-5988), whereas the purification starting from the tosylate precursor was tedious and time consuming resulting in a low yield.

In contrast to this teaching of the prior art, we found advantages of tosylate and further arylsulfonate precursors for 4-[(E)-2-(4-{2-[2-(2-[F-18]fluoroethoxy)ethoxy]ethoxy}phenyl)vinyl]-N-methylaniline compared to the corresponding mesylate. Less non-radioactive by-products that eluted close to the retention time of 4-[(E)-2-(4-{2-[2-(2-[F-18]fluoroethoxy)ethoxy]ethoxy}phenyl)vinyl]-N-methylaniline were found in the crude products if arylsulfonate precursors were used (Example 2-Example 6) compared to the crude mixture that was obtained after conversion of the mesylate precursor (Example 1).

The favorable by-product profile after radiolabeling of tosylate precursor 2b (FIG. 10) compared to the radiolabeling of mesylate precursor 2a (FIG. 7) supported an improved cartridge based purification (Example 8, Example 9).

### SUMMARY OF THE INVENTION

The present invention provides compound of Formula II for production of radiolabeled compound of Formula I and suitable salts of an inorganic or organic acid thereof, hydrates, complexes, esters, amides, solvates and prodrugs thereof and a optionally a pharmaceutically acceptable carrier, diluent, adjuvant or excipient.
The method comprises the steps of:
   Radiofluorination of compound of Formula II
   Optionally, cleavage of the protecting group
   Purification and Formulation of compound of Formula I

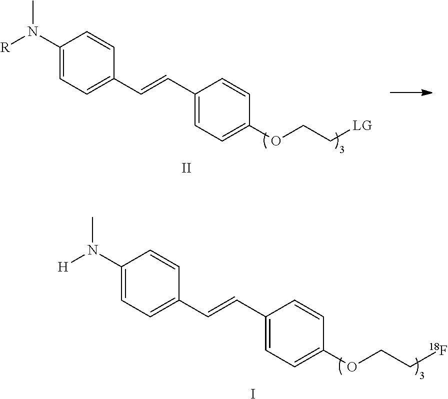

The present invention also provides compositions comprising a radiolabeled compound of Formula I or suitable salts of an inorganic or organic acid thereof, hydrates, complexes, esters, amides, solvates and prodrugs thereof and optionally a pharmaceutically acceptable carrier, diluent, adjuvant or excipient.
The present invention also provides a Kit for preparing a radiopharmaceutical preparation by the herein described process, said Kit comprising a sealed vial containing a predetermined quantity of the compound of Formula II.

### DESCRIPTION OF THE INVENTION

In a first aspect the present invention is directed to a compound of Formula II

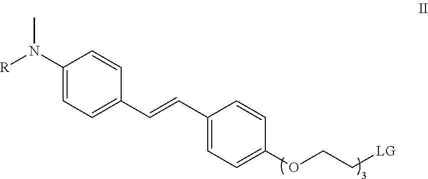

wherein:
R is selected from the group comprising

### a) H,

### b) PG.

PG is an “amine-protecting group”.

In a preferred embodiment, PG is selected from the group comprising:

### a) Boc,

### b) Trityl and

### c) 4-Methoxytrityl.

More preferably, PG is Boc.

### LG is Arylsulfonyloxy.

In a preferred embodiment LG is contains 0-3 fluorine atoms.

In a preferred embodiment Arylsulfonyloxy is selected from the group consisting of:

p-Toluenesulfonyloxy, 4-Cyanophenylsulfonyloxy, 4-Bromophenylsulfonyloxy, 4-Nitrophenylsulfonyloxy, 2-Nitrophenylsulfonyloxy, 4-Isopropyl-phenylsulfonyloxy, 2,4,6-Triisopropyl-phenylsulfonyloxy, 2,4,6-Trimethylphenylsulfonyloxy, 4-tert-Butyl-phenylsulfonyloxy, 4-Adamantylphenylsulfonyloxy and 4-Methoxyphenylsulfonyloxy.

In a more preferred embodiment, Arylsulfonyloxy is selected from the group comprising:

a) p-Toluenesulfonyloxy

### b) (2-Nitrophenyl)sulfonyloxy,

### c) (4-Cyanophenyl)sulfonyloxy

### d) (4-Bromophenyl)sulfonyloxy,

### e) (4-Adamantylphenyl)sulfonyloxy.

In a second aspect the present invention is directed to a method for producing compound of Formula I by reacting compound of Formula II

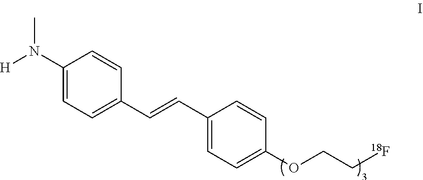

comprising the steps of:
Step 1: Radiolabeling of compound of Formula II with a F-18 fluorinating agent, to obtain compound of Formula I, if R═H or to obtain compound of Formula III, if R=PG

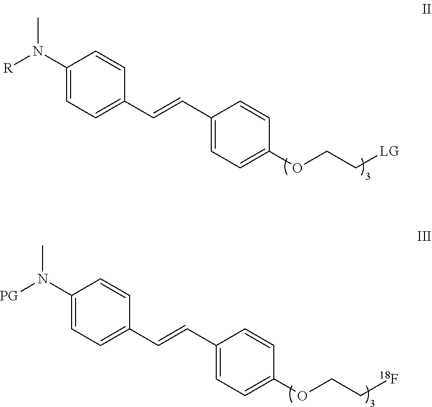

Step 2: Optionally, if R=PG, cleavage of the protecting group PG to obtain compound of Formula I
Step 3: Purification and Formulation of compound of Formula I
   wherein compound of Formula II is described above.

Step 1 comprises a straight forward [F-18]fluoro labeling reaction from compounds of Formula II for obtaining compound of Formula I (if R═H) or compound of Formula III (if R=PG).

The radiolabeling method comprises the step of reacting a compound of Formula II with a F-18 fluorinating agent for obtaining a compound of Formula III or of Formula I In a preferred embodiment, the [F-18]fluoride derivative is 4,7,13,16,21,24-Hexaoxa-1,10-diazabicyclo[8.8.8]-hexacosane K[F-18]F (crownether salt Kryptofix K[F-18]F), K[F-18]F, H[F-18]F, KH[F-18]F₂, Cs[F-18]F, Na[F-18]F or tetraalkylammonium salt of [F-18]F (e.g. [F-18]tetrabutylammonium fluoride). More preferably, the fluorination agent is K[F-18]F, H[F-18]F, [F-18]tetrabutylammonium fluoride, Cs[F-18]F or KH[F-18]F₂, most preferably K[F-18], Cs[F-18]F or [F-18]tetrabutylammonium fluoride.

The radiofluorination reactions are carried out in acetonitrile, dimethylsulfoxide or dimethylformamide or a mixture thereof. But also other solvents can be used which are well known to someone skilled in the art. Water and/or alcohols can be involved in such a reaction as co-solvent. The radiofluorination reactions are conducted for less than 60 minutes. Preferred reaction times are less than 30 minutes. Further preferred reaction times are less than 15 min. This and other conditions for such radiofluorination are known to experts (Coenen, Fluorine-18 Labeling Methods: Features and Possibilities of Basic Reactions, (2006), in: Schubiger P. A., Friebe M., Lehmann L., (eds), PET-Chemistry—The Driving Force in Molecular Imaging. Springer, Berlin Heidelberg, pp. 15-50).

In a preferred embodiment, the Radiofluorination of compound of Formula II is carried out in acetonitrile or in a mixture of acetonitrile and co-solvents, wherein the ratio of acetonitrile is at least 50%, more preferably 70%, even more preferably 90%.

In one embodiment, 1.5-75 μmol, preferably 7.5-40 μmol, more preferably 10-30 μmol, and even more preferably 12-25 μmol of compound of Formula II are used in Step 1.

In an other embodiment, 1.5-50 μmol/mL, preferably 5-25 μmol/mL, more preferably 7-20 μmol/mol of a solution of compound of Formula II in acetonitrile or an acetonitrile/co-solvent mixture are used in Step 1.

Optionally, if R=PG, Step 2 comprises the deprotection of compound of Formula III to obtain compound of Formula I. Reaction conditions are known or obvious to someone skilled in the art, which are chosen from but not limited to those described in the textbook Greene and Wuts, Protecting groups in Organic Synthesis, third edition, page 494-653, included herewith by reference. Preferred reaction conditions are addition of an acid and stirring at 0° C.-180° C.; addition of an base and heating at 0° C.-180° C.; or a combination thereof.

Preferably the Step 1 and Step 2 are performed in the same reaction vessel.

Step 3 comprises the purification and formulation of compound of Formula I. Methods for purification of radiotracers are well known to person skilled in the art and include HPLC methods as well as solid-phase extraction methods.

In one embodiment, the crude product mixture is purified by HPLC and the collected product fraction is further passed through a solid-phase cartridge to remove the HPLC solvent (such as acetonitrile) and to provide the compound of Formula I in an injectable Formulation.

In an other embodiment, the crude product mixture is purified by HPLC, wherein, the HPLC solvent mixture (e.g. mixtures of ethanol and aqueous buffers) can be part of the injectable Formulation of compound of Formula I. The collected product fraction can be diluted or mixed with other parts of the Formulation.

In an other embodiment, the crude product mixture is purified by solid-phase cartridges.

In a preferred embodiment, the method is carried out by use of a module (review: Krasikowa, Synthesis Modules and Automation in F-18 labeling (2006), in: Schubiger P. A., Friebe M., Lehmann L., (eds), PET-Chemistry—The Driving Force in Molecular Imaging. Springer, Berlin Heidelberg, pp. 289-316) which allows an automated synthesis. More preferably, the process is carried out by use of an one-pot module. Even more preferable, the process is carried out on commonly known non-cassette type modules (e.g. Ecker&Ziegler Modular-Lab, GE tracerlab FX, Raytest SynChrom) and cassette type modules (e.g. GE Tracerlab MX, GE Fastlab, IBA Synthera, Eckert&Ziegler Modular-Lab PharmTracer), optionally, further equipment such as HPLC or dispensing devices are attached to the said modules.

In a third aspect the present invention is directed to a fully automated and/or remote controlled method for production of compound of Formula I comprising the Steps and compounds as disclosed above.

In a preferred embodiment this method is a fully automated process, compliant with GMP guidelines, that provides a Formulation of Formula I for the use of administration (injection) into human.

In a fourth aspect the present invention is directed to a method for production of compounds of Formula II (with the provisio that R=PG) by reacting a compound of Formula IV with arylsulfonylhalides or arylsulfonic acid anhydrides or arylsulfonic acid, preferably with arylsulfonylhalides or arylsulfonic acid anhydrides. The formation of compounds of Formula II from compound of Formula IV can be mediated by a base or a coupling reagent as known to person skilled in the art.

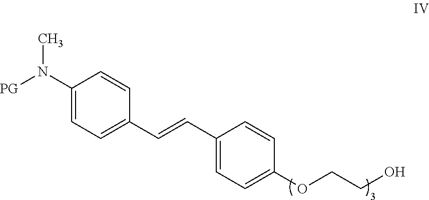

wherein PG is described above.

In a fifth aspect the present invention is directed to a method for production of compounds of Formula II (with the provisio that R═H) by reacting a compound of Formula IV with arylsulfonylhalides or arylsulfonic acid anhydrides or arylsulfonic acid, preferably with arylsulfonylhalides or arylsulfonic acid anhydrides. The formation of compounds of Formula II from compound of Formula IV can be mediated by a base or coupling reagent as known to person skilled in the art.

Subsequent cleavage of PG leads to compounds of Formula II.

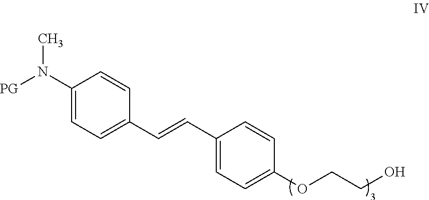

wherein PG is described above.

In a sixth aspect the present invention is directed to a Kit for the production of a pharmaceutical composition of compound of Formula I.

In one embodiment the Kit comprising a sealed vial containing a predetermined quantity of the compound of Formula II as disclosed in the first aspect.

Preferably, the Kit contains 1.5-75 μmol, preferably 7.5-50 μmol, more preferably 10-30 μmol, and more preferably 12-25 μmol of compound of Formula II.

Optionally, the Kit contains further components for manufacturing of compound of Formula I, such as solvents, solid-phase extraction cartridges, reagent for fluorination (as described above), reagent for cleavage of deprotection group, solvent or solvent mixtures for purification, solvents and excipient for formulation.

In one embodiment, the Kit contains a platform (e.g. cassette) for a “cassette-type module” (such as Tracerlab MX or IBA Synthera).

### DEFINITIONS

In the context of the present invention, preferred salts are pharmaceutically suitable salts of the compounds according to the invention. The invention also comprises salts which for their part are not suitable for pharmaceutical applications, but which can be used, for example, for isolating or purifying the compounds according to the invention.

Pharmaceutically suitable salts of the compounds according to the invention include acid addition salts of mineral acids, carboxylic acids and sulphonic acids, for example salts of hydrochloric acid, hydrobromic acid, sulphuric acid, phosphoric acid, methanesulphonic acid, ethanesulphonic acid, toluenesulphonic acid, benzenesulphonic acid, naphthalene disulphonic acid, acetic acid, trifluoroacetic acid, propionic acid, lactic acid, tartaric acid, malic acid, citric acid, fumaric acid, maleic acid and benzoic acid.

Pharmaceutically suitable salts of the compounds according to the invention also include salts of customary bases, such as, by way of example and by way of preference, alkali metal salts (for example sodium salts and potassium salts), alkaline earth metal salts (for example calcium salts and magnesium salts) and ammonium salts, derived from ammonia or organic amines having 1 to 16 carbon atoms, such as, by way of example and by way of preference, ethylamine, diethylamine, triethylamine, ethyldiisopropylamine, monoethanolamine, diethanolamine, triethanolamine, dicyclohexylamine, dimethylaminoethanol, procaine, diben-zylamine, N methylmorpholine, arginine, lysine, ethylenediamine and N methylpiperidine.

The term halogen or halo refers to Cl, Br, F or I.

The term “Arylsulfonyloxy” refers to

—O—S(O)₂-Q wherein Q is optionally substituted aryl.

The term “aryl” as employed herein by itself or as part of another group refers to monocyclic or bicyclic aromatic groups containing from 6 to 10 carbons in the ring portion, such as phenyl, naphthyl or tetrahydronaphthyl.

Whenever the term “substituted” is used, it is meant to indicate that one or more hydrogens on the atom indicated in the expression using “substituted” is/are replaced by one ore multiple moieties from the group comprising halogen, nitro, cyano, trifluoromethyl, alkyl and O-alkyl, provided that the regular valency of the respective atom is not exceeded, and that the substitution results in a chemically stable compound, i.e. a compound that is sufficiently robust to survive isolation to a useful degree of purity from a reaction mixture.

The term “alkyl” as employed herein by itself or as part of another group refers to a C₁-C₁₀ straight chain or branched alkyl group such as, for example methyl, ethyl, propyl, isopropyl, butyl, isobutyl, tert-butyl, pentyl, isopentyl, neopentyl, heptyl, hexyl, decyl or adamantyl. Preferably, alkyl is C₁-C₆ straight chain or branched alkyl or C₇-C₁₀ straight chain or branched alkyl. Lower alkyl is a C₁-C₆ straight chain or branched alkyl.

The term “amine-protecting group” as employed herein by itself or as part of another group is known or obvious to someone skilled in the art, which is chosen from but not limited to a class of protecting groups namely carbamates, amides, imides, N-alkyl amines, N-aryl amines, imines, enamines, boranes, N—P protecting groups, N-sulfenyl, N-sulfonyl and N-silyl, and which is chosen from but not limited to those described in the textbook Greene and Wuts, Protecting groups in Organic Synthesis, third edition, page 494-653, included herewith by reference. The amine-protecting group is preferably Carbobenzyloxy (Cbz), p-Methoxybenzyl carbonyl (Moz or MeOZ), tert-Butyloxycarbonyl (BOC), 9-Fluorenylmethyloxycarbonyl (FMOC), Benzyl (Bn), p-Methoxybenzyl (PMB), 3,4-Dimethoxybenzyl (DMPM), p-methoxyphenyl (PMP) or the protected amino group is a 1,3-dioxo-1,3-dihydro-2H-isoindol-2-yl (phthalimido) or an azido group.

The term “leaving group” as employed herein by itself or as part of another group is known or obvious to someone skilled in the art, and means that an atom or group of atoms is detachable from a chemical substance by a nucleophilic agent. Examples are given e.g. in Synthesis (1982), p. 85-125, table 2 (p. 86; (the last entry of this table 2 needs to be corrected: “n-C₄F₉S(O)₂—O— nonaflat” instead of “n-C₄H₉S(O)₂—O— nonaflat”), Carey and Sundberg, Organische Synthese, (1995), page 279-281, table 5.8; or Netscher, Recent Res. Dev. Org. Chem., 2003, 7, 71-83, scheme 1, 2, 10 and 15 and others). (Coenen, Fluorine-18 Labeling Methods: Features and Possibilities of Basic Reactions, (2006), in: Schubiger P. A., Friebe M., Lehmann L., (eds), PET-Chemistry—The Driving Force in Molecular Imaging. Springer, Berlin Heidelberg, pp. 15-50, explicitly: scheme 4 pp. 25, scheme 5 pp 28, table 4 pp 30, Fig 7 pp 33).

Unless otherwise specified, when referring to the compounds of Formula the present invention per se as well as to any pharmaceutical composition thereof the present invention includes all of the hydrates, salts, and complexes.

The term “F-18” means fluorine isotope ¹⁸F. The term“F-19” means fluorine isotope ¹⁹F.

The term “F-18” means fluorine isotope ¹⁸F. The term“F-19” means fluorine isotope ¹⁹F.

### EXAMPLES

### Example 1

### Radiolabeling of Mesylate Precursor 2a

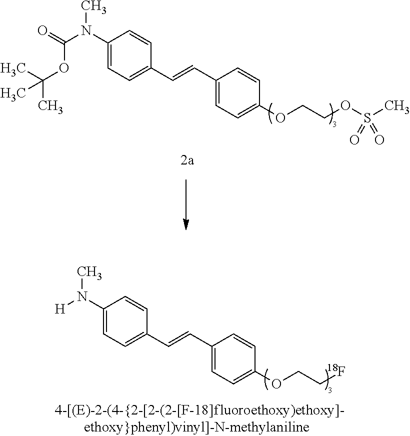

Radiolabeling was performed on a remote controlled synthesis module (Tracerlab FX_{N}). Precursor 2a (2 mg) in 0.5 mL DMSO+0.5 mL acetonitrile was treated with dried potassium carbonate/kryptofix/[F-18]fluoride complex for 6 min at 100° C. 1M HCl (1 mL)+10 mg ascorbic acid was added and the mixture was heated for 4 min at 100° C. 2M NaOH (0.5 mL), water (6 mL) and ethanol (1 mL) were added and the crude mixture was trapped on a C18 cartridge. The crude product mixture was eluted with acetonitrile and diluted with 0.1M ammonium formiat buffer (1 mL)+5 mg ascorbic acid. A sample of the crude product was taken and analyzed by analytical HPLC (FIG. 1). After purification by semi-preparative HPLC, the product was diluted with water+5 mg ascorbic acid, trapped on a C18 cartridge and eluted with 1 mL ethanol.

Yield of 4-[(E)-2-(4-{2-[2-(2-[F-18]fluoroethoxy)ethoxy]ethoxy}phenyl)-vinyl]-N-methylaniline: 21% (corrected for decay).

### Example 2

### Synthesis and Radiolabeling of Tosylate Precursor 2b

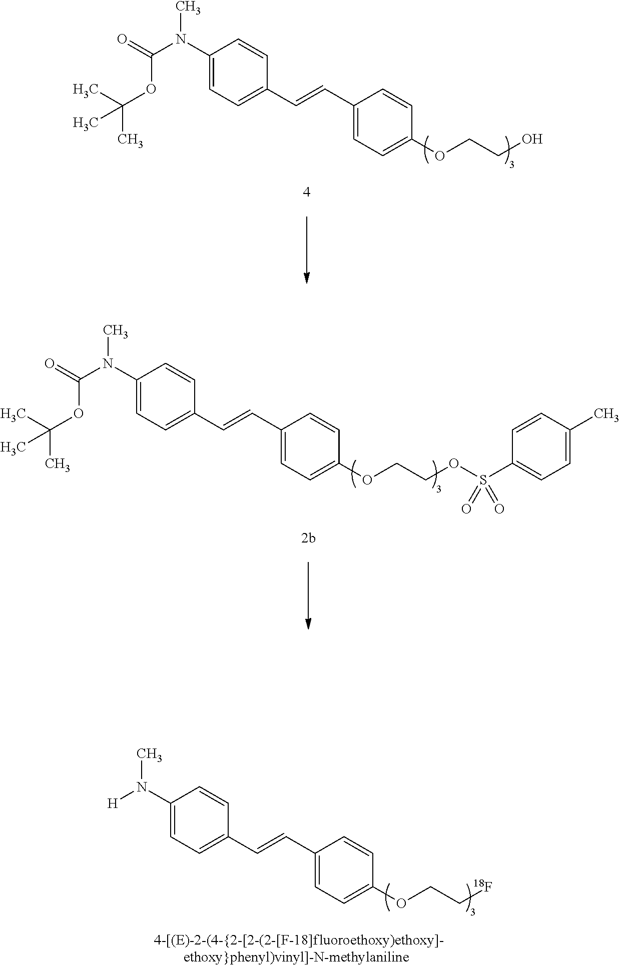

4-Dimethylaminopyridine (26.7 mg) and triethylamine (225 μL) were added to a solution of 1.0 g tert-butyl {4-[(E)-2-(4-{2-[2-(2-hydroxyethoxy)ethoxy]ethoxy}phenyl)vinyl]phenyl}methylcarbamate (4) in dichloromethane (12 mL) at 0° C. A solution of p-toluenesulfonyl chloride (417 mg) in dichloromethane (13.5 mL) was added at 0° C. The resulting mixture was stirred at room temperature over night. The solvent was removed under reduced pressure and the crude product was purified by flash chromatography (silica, 0-80% ethyl acetate in hexane). 850 mg 2b were obtained as colorless solid.

¹H NMR (300 MHz, CDCl3) δ ppm 1.46 (s, 9H), 2.43 (s, 3H), 3.27 (s, 3H), 3.59-3.73 (m, 6H), 3.80-3.86 (m, 2H), 4.05-4.19 (m, 2H), 6.88-7.05 (m, 4H), 7.21 (d, J=8.3 Hz, 2H), 7.32 (d, J=8.3 Hz, 2H), 7.39-7-47 (m, 4H), 7.80 (d, J=8.3 Hz, 2H).

MS (ESIpos): m/z=612 (M+H)⁺

Radiolabeling was performed on a remote controlled synthesis module (Tracerlab FX_{N}). Precursor 2b (2 mg) in 0.5 mL DMSO+0.5 mL acetonitrile was treated with dried potassium carbonate/kryptofix/[F-18]fluoride complex for 6 min at 100° C. 1M HCl (1 mL)+10 mg ascorbic acid was added and the mixture was heated for 4 min at 100° C. 2M NaOH (0.5 mL), water (6 mL) and ethanol (1 mL) were added and the crude mixture was trapped on a C18 cartridge. The crude product mixture was eluted with acetonitrile and diluted with 0.1M ammonium formiat buffer (1 mL)+5 mg ascorbic acid. A sample of the crude product was taken and analyzed by analytical HPLC (FIG. 2). After purification by semi-preparative HPLC, the product was diluted with water+5 mg ascorbic acid, trapped on a C18 cartridge and eluted with 1 mL ethanol.

Yield of 4-[(E)-2-(4-{2-[2-(2-[F-18]fluoroethoxy)ethoxy]ethoxy}phenyl)-vinyl]-N-methylaniline: 25% (corrected for decay).

### Example 3

### Synthesis and Radiolabeling of 2c (2-[2-(2-{4-[(E)-2-{4-[(tert-butoxycarbonyl)(methyl)amino]phenyl}vinyl]phenoxy}ethoxy)ethoxy]ethyl 4-bromobenzenesulfonate)

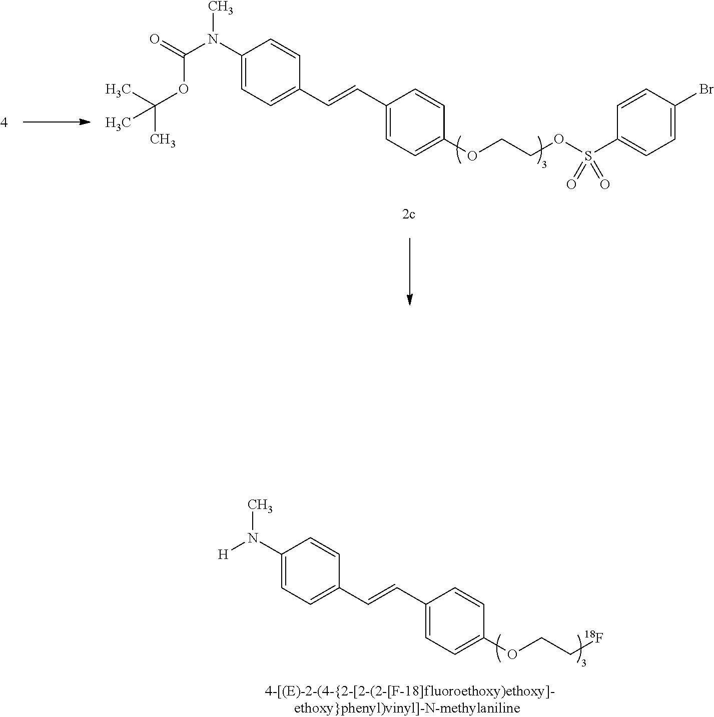

To a stirred solution of 100 mg (0,219 mmol) tert-butyl-{4-[(E)-2-(4-{2-[2-(2-hydroxyethoxy)ethoxy]ethoxy}phenyl)vinyl]phenyl}methylcarbamate (WO2006/066104) in 2 mL THF was added a solution of 140 mg (0.548 mmol) 4-brombenzene sulfonylchlorid in 3 mL THF drop by drop. The reaction mixture was cooled to 0° C. 156.8 mg (1.1 mmol) potassium trimethylsilanolat was added. The milky suspension was stirred at 0° C. for 2 hours and at 80° C. for another 2 hours. The reaction mixture was poured onto ice-cooled water. The aqueous solution was extracted with dichloromethane several times. The combined organic phases were dried with sodium sulphate and concentrated in vacuum. The crude product was purified using silica gel with ethyl acetate/hexane-gradient as mobile phase. The desired product 2c was obtained with 77 mg (0.114 mmol, 52.0% yield).

¹H NMR (300 MHz, CDCl₃) δ ppm 1.39 (s, 10H) 3.20 (s, 3H) 3.50-3.57 (m, 2H) 3.57-3.61 (m, 2H) 3.61-3.66 (m, 2H) 3.72-3.80 (m, 2H) 4.02-4.10 (m, 2H) 4.10-4.17 (m, 2H) 6.79-6.85 (m, 2H) 6.91 (d, J=8.10 Hz, 2H) 7.10-7.17 (m, 2H) 7.32-7.41 (m, 5H) 7.57-7.65 (m, 2H) 7.67-7.74 (m, 2H)

MS (ESIpos): m/z=676/678 (M+H)⁺

Radiolabeling was performed on a remote controlled synthesis module (Tracerlab FX_{N}). Precursor 2c (2 mg) in 0.5 mL DMSO+0.5 mL acetonitrile was treated with dried potassium carbonate/kryptofix/[F-18]fluoride complex for 6 min at 100° C. 1M HCl (1 mL)+10 mg ascorbic acid was added and the mixture was heated for 4 min at 100° C. 2M NaOH (0.5 mL), water (6 mL) and ethanol (1 mL) were added and the crude mixture was trapped on a C18 cartridge. The crude product mixture was eluted with acetonitrile and diluted with 0.1M ammonium formiat buffer (1 mL)+5 mg ascorbic acid. A sample of the crude product was taken and analyzed by analytical HPLC (FIG. 3). After purification by semi-preparative HPLC, the product was diluted with water+5 mg ascorbic acid, trapped on a C18 cartridge and eluted with 1 mL ethanol.

Yield of 4-[(E)-2-(4-{2-[2-(2-[F-18]fluoroethoxy)ethoxy]ethoxy}phenyl)-vinyl]-N-methylaniline: 43% (corrected for decay).

### Example 4

### Synthesis and Radiolabeling of 2d (2-[2-(2-{4-[(E)-2-{4-[(tert-butoxycarbonyl)(methyl)amino]phenyl}vinyl]phenoxy}ethoxy)ethoxy]ethyl 4-(adamantan-1-yl)benzenesulfonate)

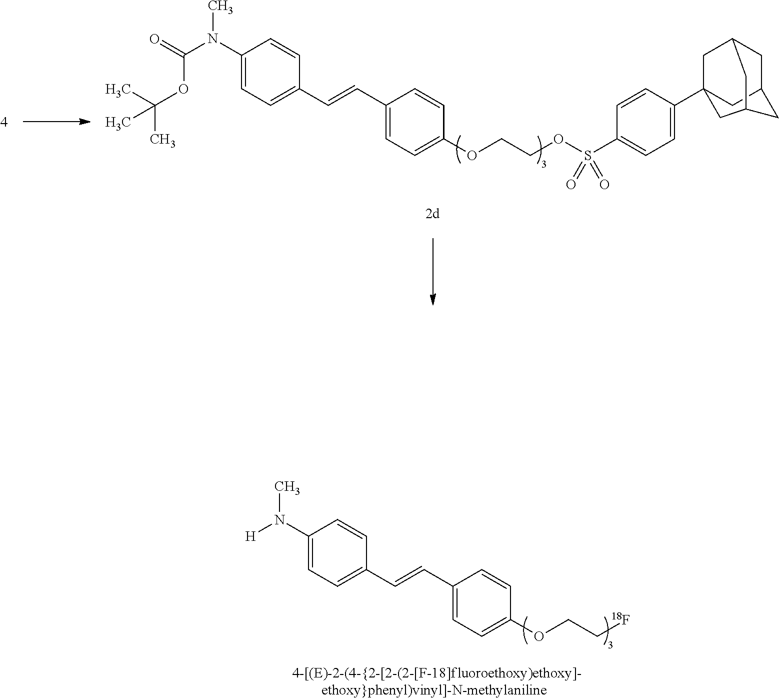

To a stirred solution of 151 mg (0.330 mmol) tert-butyl-{4-[(E)-2-(4-{2-[2-(2-hydroxyethoxy)ethoxy]ethoxy}phenyl)vinyl]phenyl}methylcarbamate (WO2006/066104), 4.03 mg (0,033 mmol) DMAP und 36.7 mg (363 mmol) triethylamine in 4 mL dichlormethane was added a solution of 97.4 mg (0,313 mmol) 4-(adamantan-1-yl)benzene sulfonylchloride in 1 mL dichlormethane at 0° C. The reaction mixture was stirred at 0° C. for 1 hour and over night at room temperature. 7.3 mg (0,072 mmol) triethylamin und 19.5 mg (0.062 mmol) 4-(adamantan-1-yl)benzenesulfonyl chloride were added to the reaction mixture. The reaction mixture was stirred at room temperature for 3 days. It was concentrated in vacuum. The crude product was purified using silica gel with ethyl acetate/hexane-gradient as mobile phase. The desired product 2d was obtained with 104 mg (0.142 mmol, 43.4% yield).

¹H NMR (300 MHz, CDCl₃) δ ppm 1.51 (s, 9H), 1.62 (s, 1H), 1.74-1.91 (m, 6H), 1.94 (d, J=3.20 Hz, 6H), 2.16 (br. s., 3H), 3.31 (s, 3H), 3.63-3.69 (m, 2H), 3.69-3.73 (m, 2H), 3.76 (dd, J=5.27, 4.52 Hz, 2H), 3.89 (d, J=4.90 Hz, 2H), 4.13-4.26 (m, 4H), 6.95 (d, J=8.85 Hz, 2H), 7.02 (d, J=8.29 Hz, 2H), 7.25 (d, J=8.48 Hz, 2H), 7.40-7.52 (m, 4H), 7.55 (m, J=8.67 Hz, 2H), 7.89 (m, J=8.67 Hz, 2H)

MS (ESIpos): m/z=732 (M+H)⁺

Radiolabeling was performed on a remote controlled synthesis module (Tracerlab FX_{N}). Precursor 2d (2 mg) in 0.5 mL DMSO+0.5 mL acetonitrile was treated with dried potassium carbonate/kryptofix/[F-18]fluoride complex for 6 min at 100° C. 1M HCl (1 mL)+10 mg ascorbic acid was added and the mixture was heated for 4 min at 100° C. 2M NaOH (0.5 mL), water (6 mL) and ethanol (1 mL) were added and the crude mixture was trapped on a C18 cartridge. The crude product mixture was eluted with acetonitrile and diluted with 0.1M ammonium formiat buffer (1 mL)+5 mg ascorbic acid. A sample of the crude product was taken and analyzed by analytical HPLC (FIG. 4). After purification by semi-preparative HPLC, the product was diluted with water+5 mg ascorbic acid, trapped on a C18 cartridge and eluted with 1 mL ethanol.

Yield of 4-[(E)-2-(4-{2-[2-(2-[F-18]fluoroethoxy)ethoxy]ethoxy}phenyl)-vinyl]-N-methylaniline: 27% (corrected for decay).

### Example 5

### Synthesis and radiolabeling of 2e (2-[2-(2-{4-[(E)-2-{4-[(tert-butoxycarbonyl)(methyl)amino]phenyl}vinyl]phenoxy}ethoxy)ethoxy]ethyl 4-cyanobenzenesulfonate)

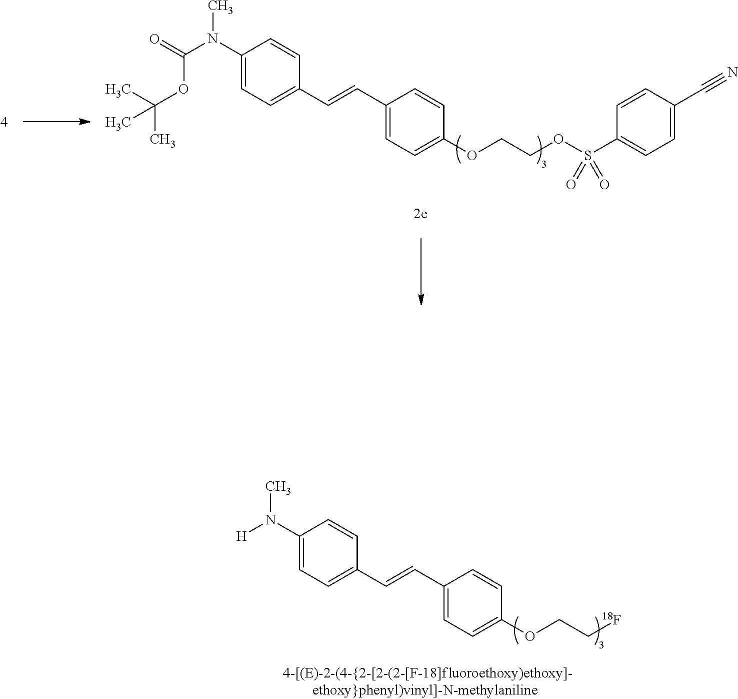

To a stirred solution of 151 mg (0.330 mmol) tert-butyl-{4-[(E)-2-(4-{2-[2-(2-hydroxyethoxy)ethoxy]ethoxy}phenyl)vinyl]phenyl}methylcarbamate (WO2006/066104), 4.03 mg (0.033 mmol) DMAP and 36.7 mg (0.363 mmol) triethylamine in 4 mL dichlormethane was added a solution of 63.2 mg (0.313 mmol) 4-cyanobenzenesulfonyl chloride in 1 mL dichlormethane at 0° C. The reaction mixture was stirred over night and concentrated in vacuum. The crude product was purified using silica gel with ethyl acetate/hexane-gradient as mobile phase. The desired product 2e was obtained with 118 mg (0.190 mmol, 57.6% yield).

¹H NMR (400 MHz, CDCl₃) δ ppm 1.47 (s, 9H) 3.28 (s, 3H) 3.58-3.63 (m, 2H) 3.63-3.68 (m, 2H) 3.70-3.75 (m, 2H) 3.81-3.87 (m, 2H) 4.11-4.18 (m, 2H) 4.24-4.30 (m, 2H) 6.91 (d, J=8.59 Hz, 2H) 6.99 (dt, 2H) 7.22 (d, J=8.34 Hz, 2H) 7.39-7.50 (m, 4H) 7.83 (m, J=8.59 Hz, 2H) 7.98-8.11 (m, 2H)

MS (ESIpos): m/z=623 (M+H)⁺

Radiolabeling was performed on a remote controlled synthesis module (Tracerlab FX_{N}). Precursor 2e (2 mg) in 0.5 mL DMSO+0.5 mL acetonitrile was treated with dried potassium carbonate/kryptofix/[F-18]fluoride complex for 6 min at 100° C. 1M HCl (1 mL)+10 mg ascorbic acid was added and the mixture was heated for 4 min at 100° C. 2M NaOH (0.5 mL), water (6 mL) and ethanol (1 mL) were added and the crude mixture was trapped on a C18 cartridge. The crude product mixture was eluted with acetonitrile and diluted with 0.1M ammonium formiat buffer (1 mL)+5 mg ascorbic acid. A sample of the crude product was taken and analyzed by analytical HPLC (FIG. 5). After purification by semi-preparative HPLC, the product was diluted with water+5 mg ascorbic acid, trapped on a C18 cartridge and eluted with 1 mL ethanol.

Yield of 4-[(E)-2-(4-{2-[2-(2-[F-18]fluoroethoxy)ethoxy]ethoxy}phenyl)-vinyl]-N-methylaniline: 31% (corrected for decay).

### Example 6

### Synthesis and radiolabeling of 2f (2-[2-(2-{4-[(E)-2-{4-[(tert-butoxycarbonyl)(methyl)amino]phenyl}vinyl]phenoxy}ethoxy)ethoxy]ethyl 2-nitrobenzenesulfonate)

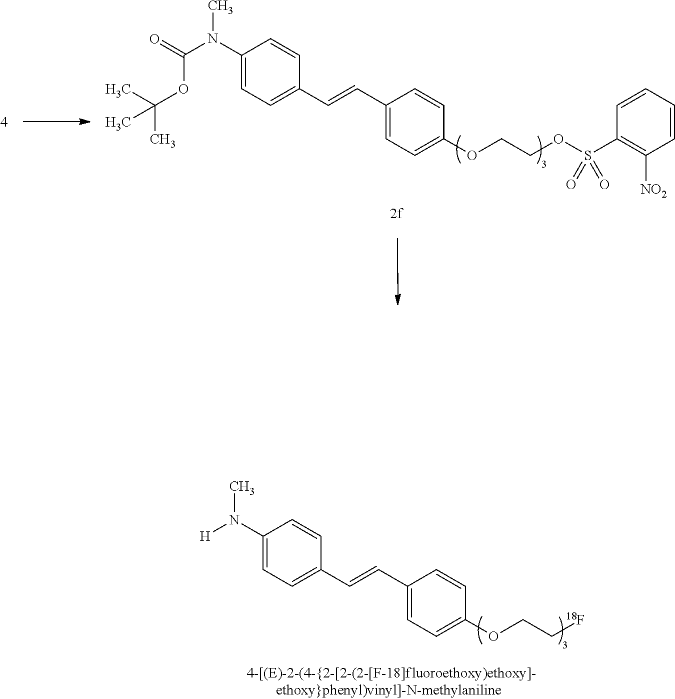

To a stirred solution of 200 mg (0.437 mmol) tert-butyl-{4-[(E)-2-(4-{2-[2-(2-hydroxyethoxy)ethoxy]ethoxy}phenyl)vinyl]phenyl}methylcarbamate (WO2006/066104), 5.34 mg (0.044 mmol) DMAP and 47.5 mg (0.470 mmol) triethylamine in 4 mL dichlormethane was added a solution of 92 mg (0,415 mmol) 2-nitrobenzenesulfonyl chloride in 1 mL dichlormethane at 0° C. The reaction mixture was stirred over night and concentrated in vacuum. The crude product was purified with ethyl acetate/hexane-gradient as mobile phase using silica gel. The desired product 2f was obtained with 77 mg (0.119 mmol, 59.5 yield).

¹H NMR (400 MHz, CDCl₃) δ ppm 1.39 (s, 9H) 3.20 (s, 3H) 3.55-3.63 (m, 4H) 3.59 (m, 4H) 3.69-3.74 (m, 2H) 3.75-3.80 (m, 2H) 4.06 (dd, J=5.68, 3.92 Hz, 2H) 4.32-4.37 (m, 2H) 6.80-6.84 (m, 2H) 6.84-6.98 (dt, 2H) 7.14 (d, J=8.59 Hz, 2H) 7.35 (d, J=3.03 Hz, 2H) 7.37 (d, J=2.78 Hz, 2H) 7.62-7.74 (m, 3H) 8.06-8.11 (m, 1H)

Radiolabeling was performed on a remote controlled synthesis module (Tracerlab FX_{N}). Precursor 2e (2 mg) in 0.5 mL DMSO+0.5 mL acetonitrile was treated with dried potassium carbonate/kryptofix/[F-18]fluoride complex for 6 min at 100° C. 1M HCl (1 mL)+10 mg ascorbic acid was added and the mixture was heated for 4 min at 100° C. 2M NaOH (0.5 mL), water (6 mL) and ethanol (1 mL) were added and the crude mixture was trapped on a C18 cartridge. The crude product mixture was eluted with acetonitrile and diluted with 0.1M ammonium formiat buffer (1 mL)+5 mg ascorbic acid. A sample of the crude product was taken and analyzed by analytical HPLC (FIG. 6). After purification by semi-preparative HPLC, the product was diluted with water+5 mg ascorbic acid, trapped on a C18 cartridge and eluted with 1 mL ethanol.

Yield of 4-[(E)-2-(4-{2-[2-(2-[F-18]fluoroethoxy)ethoxy]ethoxy}phenyl)-vinyl]-N-methylaniline: 21% (corrected for decay).

### Example 7

### Radiolabeling of Mesylate 2a and Cartridge Based Purification

The synthesis was performed on a Tracerlab MX synthesizer. [F-18]Fluoride (2.0 GBq) was trapped on a QMA cartridge (Waters). The activity was eluted into the reactor using an eluent mixture (22 mg kryptofix, 0.7 mL methanol, 0.1 mL 0.2M potassium mesylate solution, 0.01 mL tetrabutylammonium bicarbonate solution). The mixture was dried (heating, nitrogen stream, vacuum, addition of acetonitrile) and 7.0 mg precursor 2a in 1.5 mL tert-amyl alcohol+0.3 mL acetonitrile were added to the residue. After heating for 20 min at 120° C., the solvent was evaporated and a mixture of 2.2 mL 1.5M HCl, 1.1 mL acetonitrile and 30 mg sodium ascorbate was added. The resulting solution was heated for 7.5 min at 100° C. The crude product (910 MBq, 64% corrected for decay) was transferred to a vial and diluted with 1.5 mL 2M NaOH and 0.3 mL 0.1M ammonium formiate solution. A sample was analyzed by analytical HPLC (FIG. 7). The crude product was loaded on a Chromabond Flash cartridge (RS 4 Nucleodur 100-30 C18ec, Macherey-Nagel) and 40% EtOH in phosphate buffer (pH 7.4) were flushed through the cartridge by a HPLC pump with 9 mL/min. A radioactivity and a UV detector were attached to the HPLC to monitor the purification (FIG. 8). After 15 min the solvent was changed to 50% EtOH in phosphate buffer (pH 7.4). The product fraction (25% decay corrected) was collected from 17.5-19 min and analyzed by analytical HPLC (FIG. 9).

### Example 8

### Radiolabeling of Tosylate 2b and Cartridge Based Purification

The synthesis was performed on a Tracerlab MX synthesizer. [F-18]Fluoride (1.6 GBq) was trapped on a QMA cartridge (Waters). The activity was eluted into the reactor using an eluent mixture (22 mg kryptofix, 0.7 mL methanol, 0.1 mL 0.2M potassium mesylate solution, 0.01 mL tetrabutylammonium bicarbonate solution). The mixture was dried (heating, nitrogen stream, vacuum, addition of acetonitrile) and 8.0 mg precursor 2b in 1.5 mL tert-amyl alcohol+0.3 mL acetonitrile were added to the residue. After heating for 20 min at 120° C., the solvent was evaporated and a mixture of 2.2 mL 1.5M HCl, 1.1 mL acetonitrile and 30 mg sodium ascorbate was added. The resulting solution was heated for 7.5 min at 100° C. The crude product (775 MBq, 67% corrected for decay) was transferred to a vial and diluted with 1.5 mL 2M NaOH and 0.3 mL 0.1M ammonium formiate solution. A sample was analyzed by analytical HPLC (FIG. 10). The crude product was loaded on a Chromabond Flash cartridge (RS 4 Nucleodur 100-30 C18ec, Macherey-Nagel) and 40% EtOH in phosphate buffer (pH 7.4) were flushed through the cartridge by a HPLC pump with 9 mL/min. A radioactivity and a UV detector were attached to the HPLC to monitor the purification (FIG. 11). After 15 min the solvent was changed to 50% EtOH in phosphate buffer (pH 7.4). The product fraction (31% decay corrected) was collected from 17.5-19 min and analyzed by analytical HPLC (FIG. 12).

### Example 9

### Radiolabeling and Cartridge Based Purification on Tracerlab MX Using Tosylate 2b

For synthesis and purification of 4-[(E)-2-(4-{2-[2-(2-[F-18]fluoroethoxy)ethoxy]-ethoxy}phenyl)vinyl]-N-methylaniline on the Tracerlab MX, a Kit was assembled (Table 1).

TABLE 1
Composition of Kit for manufacturing of
4-[(E)-2-(4-{2-[2-(2-[F-18]fluoroethoxy)ethoxy]-
ethoxy}phenyl)vinyl]-N-methylaniline on tracerlab MX

| | |
|---|---|
| Eluent vial | 22 mg kryptofix. 7 mg potassium carbonate |
| | in 300 μL water + 300 μL acetonitrile |
| Blue capped vial | 8 mL acetonitrile |
| Red capped vial | 8 mg precursor 2b |
| Green capped vial | 2 mL 1.5M HCl |
| 2 mL syringe | 1.5 mL 2M NaOH + 2 mL phosphate buffer |
| Solvent bag 1 | 40% EtOH in phosphate buffer (pH 7.4) |
| Solvent bag 2 | 50% EtOH in phosphate buffer (pH 7.4) |
| Anion exchange cartridge | QMA light, Waters (pre-conditioned) |
| Purification cartridge | Chromabond Flash RS 4 Nucleodur |
| | 100-30 C18ec, Macherey-Nagel |
| Product vial | 50 mL vial |
| Formulation basis 1 | 100 mg Ascorbic acid |
| Formulation basis 2 | 122 mg Na₂HPO₄•H₂O, 8.9 mL PEG 400, |
| | 26.1 mL water |

The setup of the cassette on the MX module is illustrated in FIG. 13. Precursor 2b was dissolved in the “red capped vial” during the synthesis sequence using approximately 1.8 mL acetonitrile from the “blue capped vial”. Fluoride (2.4 GBq) was transferred to the MX module and was trapped on the QMA cartridge. The activity was eluted into the reactor with potassium carbonate/kryptofix mixture from the “eluent vial”. After azeotropic drying (heating, vacuum, nitrogen stream and addition of acetonitrile from the “blue capped vial”), the solution of 2b in acetonitrile was transferred from the “red capped vial” into the reactor. The resulting mixture was heated for 10 min at 120° C. HCl was transferred via the syringes from the “green capped vial” into the reactor. The mixture was heated for 5 min at 110° C. During deprotection, solvent mixture from “Solvent bag 1” was flushed through the “Purification cartridge” by the left syringe. The crude product mixture was mixed with sodium hydroxid/buffer mixture from the “2 mL syringe” and diluted with the solvent 1 from “Solvent bag 1”. The diluted crude product mixture was passed through the “Purification cartridge”. To remove non-radioactive by-products, solvent 1 from “Solvent bag 1” was filled into the left syringe and flushed through the “Purification cartridge” into the waste bottle. This procedure was repeated six times. Solvent 2 from “Solvent bag 2” was filled into the right syringe and transferred to the left syringe. Solvent 2 was flushed by the left syringe through the “Purification cartridge”. The first fraction was allowed to go to the waste bottle, but a fraction of 7.5 mL was automatically collected into the right syringe. Finally, the product fraction was transferred to the product vial (that was pre-filled with “Formulation basis 1” and “Formulation basis 2”). 770 MBq (32% not corrected for decay) 4-[(E)-2-(4-{2-[2-(2-[F-18]fluoroethoxy)ethoxy]-ethoxy}phenyl)vinyl]-N-methylaniline were obtained in 58 min overall manufacturing time. The cartridge based purification provided radiochemical and chemical pure product, similar to the purity obtained by semi-preparative HPLC (FIG. 14, FIG. 15).

### Example 10

### Synthesis and Radiolabeling Unprotected Tosylate Precursors 2g

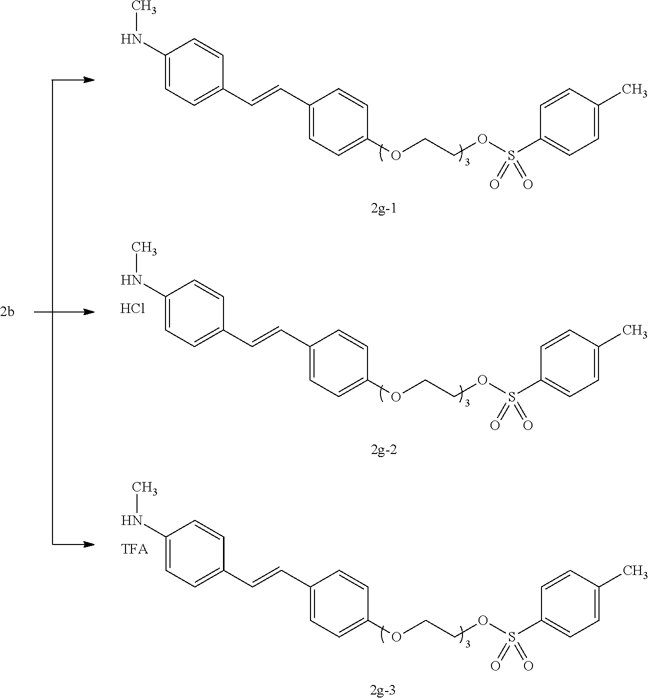

### Precursor Synthesis

### a) 2-{2-[2-(4-{(E)-2-[4-(methylamino)phenyl]vinyl}phenoxy)ethoxy]-ethoxy}ethyl 4-methylbenzenesulfonate (2g-1)

200 mg 2-[2-(2-{4-[(E)-2-{4-[(tert-butoxycarbonyl)(methyl)amino]phenyl}-vinyl]phenoxy}ethoxy)ethoxy]ethyl 4-methylbenzenesulfonate (2b) were dissolved in 2.5 mL dichloromethane. 250 μL trifluoroacetic acid were added and the mixture was stirred for 4 h at room temperature. The solvent was removed under reduced pressure. The crude product was dissolved in dichlormethane (5 mL) and washed with sodium carbonate solution (10%, 2×2 mL). The organic layer was dried over sodium sulfate, the solvent was removed under reduced pressure and the residue was purified by flash chromatography (silica, 12-100% ethyl acetate in hexane). 84 mg 2g-1 were obtained as light red solid.

¹H NMR (300 MHz, CDCl₃) δ ppm 2.42 (s, 3H), 2.87 (s, 3H), 3.61-3.64 (m, 2H), 3.65-3.68 (m, 2H), 3.69-3.72 (m, 2H), 3.81-3.84 (m, 2H), 4.10-4.13 (m, 2H), 4.15-4.17 (m, 2H), 6.63 (d, J=8.3 Hz, 2H), 6.84-6.91 (m, 4H), 7.32 (d, J=7.9 Hz, 2H), 7.34 (d, J=8.7 Hz, 2H), 7.39 (d, J=8.7 Hz, 2H), 7.80 (d, J=8.3 Hz, 2H).

MS (ESIpos): m/z=512 (M+H)⁺

### b) 2-{2-[2-(4-{(E)-2-[4-(methylamino)phenyl]vinyl}phenoxy)ethoxy]-ethoxy}ethyl 4-methylbenzenesulfonate hydrochloride (2g-2)

200 mg 2-[2-(2-{4-[(E)-2-{4-[(tert-butoxycarbonyl)(methyl)amino]phenyl}-vinyl]phenoxy}ethoxy)ethoxy]ethyl 4-methylbenzenesulfonate (2b) were dissolved in a 2M solution of HCl in diethyl ether. The mixture was stirred at room temperature for 72 h. The solvent was removed under reduced pressure. Diethyl ether was added and the precipitate was collected, washed with diethyl ether and dried under reduced pressure. 160 mg 2g-2 were obtained as light yellow solid.

¹H NMR (300 MHz, CDCl₃) δ ppm 2.43 (s, 3H), 3.03 (s, 3H), 3.62-3.64 (m, 2H), 3.66-3.68 (m, 2H), 3.69-3.72 (m, 2H), 3.82-3.85 (m, 2H), 4.12-4.14 (m, 2H), 4.16-4.18 (m, 2H), 6.88-6.94 (m, 3H), 7.04 (d, J=16.2 Hz, 1H), 7.32 (d, J=7.9 Hz, 2H), 7.42 (d, J=8.7 Hz, 2H), 7.49-7-56 (m, 4H), 7.80 (d, J=8.3 Hz, 2H).

MS (ESIpos): m/z=512 (M+H)⁺

### c) 2-{2-[2-(4-{(E)-2-[4-(methylamino)phenyl]vinyl}phenoxy)ethoxy]-ethoxy}ethyl 4-methylbenzenesulfonate trifluoroacetate (2g-3)

200 mg 2-[2-(2-{4-[(E)-2-{4-[(tert-butoxycarbonyl)(methyl)amino]phenyl}-vinyl]phenoxy}ethoxy)ethoxy]ethyl 4-methylbenzenesulfonate (2b) were dissolved in 2.5 mL dichloromethane. 252 μL trifluoroacetic acid were added and the mixture was stirred for 5 h at room temperature. The solvent was removed under reduced pressure. The crude product was washed with hexane and diethyl ether and was dried under reduced pressure. 84 mg 2g-3 were obtained as light brown solid.

¹H NMR (300 MHz, DMSO d6) δ ppm 2.40 (s, 3H), 2.72 (s, 3H), 3.46-3.50 (m, 2H), 3.51-3.55 (m, 2H), 3.57-3.61 (m, 2H), 3.69-3.73 (m, 2H), 4.10-4.09 (m, 2H), 4.10-4.13 (m, 2H),), 6.59-6.66 (m, 2H), 6.85-6.97 (m, 4H), 7.34 (d, J=8.3 Hz, 2H), 7.43 (d, J=8.8 Hz, 2H), 7.46 (d, J=8.1 Hz, 2H), 7.76 (d, J=8.3 Hz, 2H).

MS (ESIpos): m/z=512 (M+H)⁺

### Radiolabeling

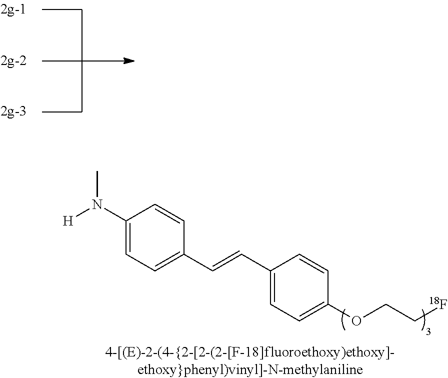

Radiolabelings have been performed using potassium carbonate/kryptofix or tetrabutylammonium hydroxide or tetrabutylammonium bicarbonate as reagent.

a) Radiolabeling with Potassium Carbonate/Kryptofix

[F-18]fluoride was trapped on a QMA cartridge. The activity was eluted using a solution of 7.5 mg kryptofix, 1 mg potassium carbonate in 1425 μL acetonitrile and 75 μL water. The mixture was dried under gentle nitrogen stream at 120° C. Drying was repeated after addition of 1 mL acetonitrile. The precursor (5.0 mg 2g-1 or 5.36 mg 2g-2 or 6.11 mg 2g-3) in 1 mg acetonitrile was added and the mixture was heated at 120° C. for 15 min. Fluoride incorporation was measured by radio-TLC (silica, ethyl acetate), results as summarized in Table 2.

b) Radiolabeling with Tetrabutylammonium Hydroxide

[F-18]fluoride was trapped on a QMA cartridge. The activity was eluted using a mixture of 300 μL≈b 4% (wt) n-Bu₄OH and 600 μL acetonitrile. The mixture was dried under gentle nitrogen stream at 120° C. Drying was repeated after addition of 1 mL acetonitrile. The precursor (5.0 mg 2g-1 or 5.36 mg 2g-2 or 6.11 mg 2g-3) in 1 mg acetonitrile was added and the mixture was heated at 120° C. for 15 min. Fluoride incorporation was measured by radio-TLC (silica, ethyl acetate), results as summarized in Table 2.

c) Radiolabeling with Tetrabutylammonium Bicarbonate

[F-18]fluoride was trapped on a QMA cartridge. The activity was eluted using a mixture of 300 μL≈4% (wt) n-Bu₄NHCO₃ (a aqueous solution of 4% n-Bu₄OH was saturated with carbon dioxide) and 600 μL acetonitrile. The mixture was dried under gentle nitrogen stream at 120° C. Drying was repeated after addition of 1 mL acetonitrile. The precursor (5.0 mg 2g-1 or 5.36 mg 2g-2 or 6.11 mg 2g-3) in 1 mL acetonitrile was added and the mixture was heated at 120° C. for 15 min. Fluoride incorporation was measured by radio-TLC (silica, ethyl acetate), results as summarized in Table 2.

TABLE 2
Radiolabelings of 2g-1, 2g-2, 2g-3

| Precursor | Reagent | F-18 incorporation |
|---|---|---|
| 5.0 mg | Potassium carbonate/kryptofix | 91% |
| 2g-1 | n-Bu₄NOH | 26% |
| | n-Bu₄NHCO₃ | 39% |
| 5.36 mg | Potassium carbonate/kryptofix | 45% |
| 2g-2 | n-Bu₄NOH | 18% |
| | n-Bu₄NHCO₃ | 75% |
| 6.11 mg | Potassium carbonate/kryptofix | 77% |
| 2g-3 | n-Bu₄NOH | 21% |
| | n-Bu₄NHCO₃ | 78% |

### DESCRIPTION OF THE FIGURES

FIG. 1 Crude product mixture after conversion of 2a; top: radioactivity channel; bottom: UV channel

FIG. 2 Crude product mixture after conversion of 2b; top: radioactivity channel; bottom: UV channel

FIG. 3 Crude product mixture after conversion of 2c; top: radioactivity channel; bottom: UV channel

FIG. 4 Crude product mixture after conversion of 2d; top: radioactivity channel; bottom: UV channel

FIG. 5 Crude product mixture after conversion of 2e; top: radioactivity channel; bottom: UV channel

FIG. 6 Crude product mixture after conversion of 2f; top: radioactivity channel; bottom: UV channel

FIG. 7 Crude product mixture after conversion of 2a on tracerlab MX; a: radioactivity channel; b: UV channel

FIG. 8 Cartridge based purification after conversion of 2a; top: radioactivity channel; bottom: UV channel

FIG. 9 4-[(E)-2-(4-{2-[2-(2-[F-18]fluoroethoxy)ethoxy]ethoxy}phenyl)vinyl]-N-methylaniline after cartridge based purification; a: radioactivity channel; b: UV channel; c: co-elution with non-radioactive reference 4-[(E)-2-(4-{2-[2-(2-fluoroethoxy)ethoxy]ethoxy}phenyl)vinyl]-N-methylaniline (UV)

FIG. 10 Crude product mixture after conversion of 2b on tracerlab MX; a: radioactivity channel; b: UV channel

FIG. 11 Cartridge based purification after conversion of 2b; a: radioactivity channel; b: UV channel

FIG. 12 4-[(E)-2-(4-{2-[2-(2-[F-18]fluoroethoxy)ethoxy]ethoxy}phenyl)vinyl]-N-methylaniline after cartridge based purification; a: radioactivity channel; be: UV channel; c: co-elution with non-radioactive reference 4-[(E)-2-(4-{2-[2-(2-fluoroethoxy)ethoxy]ethoxy}phenyl)vinyl]-N-methylaniline (UV)

FIG. 13 Setup of Tracerlab MX

FIG. 14 Analytical HPLC of crude product of MX synthesis prior passing through “Purification cartridge” (sample was taken from reactor); a: radioactivity; b: UV signal 320 nm

FIG. 15 Analytical HPLC of 4-[(E)-2-(4-{2-[2-(2-[F-18]fluoroethoxy)ethoxy]-ethoxy}phenyl)vinyl]-N-methylaniline after MX synthesis and cartridge based purification; a: radioactivity; b: UV signal 320 nm; c: co-elution with non-radioactive reference 4-[(E)-2-(4-{2-[2-(2-fluoroethoxy)ethoxy]ethoxy}phenyl)vinyl]-N-methylaniline (UV)

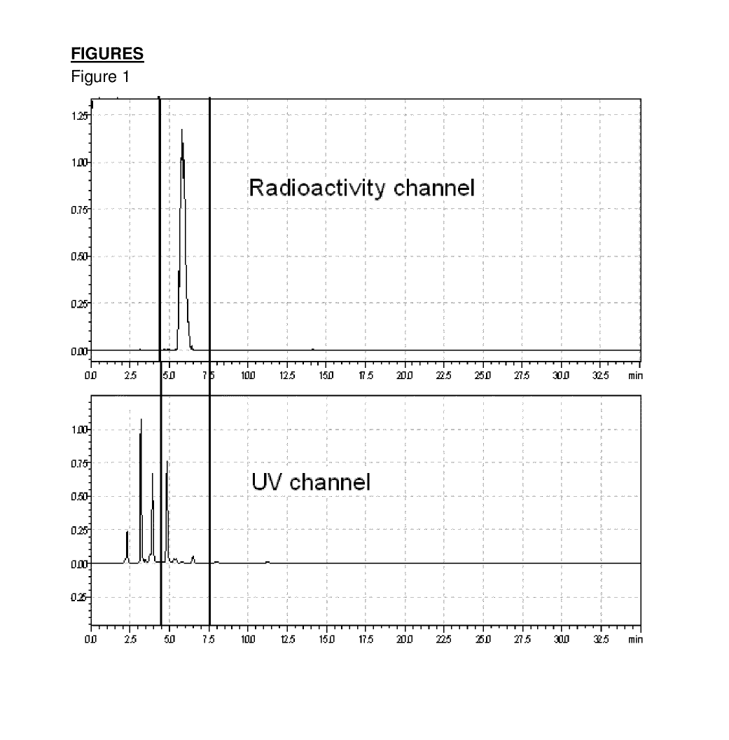

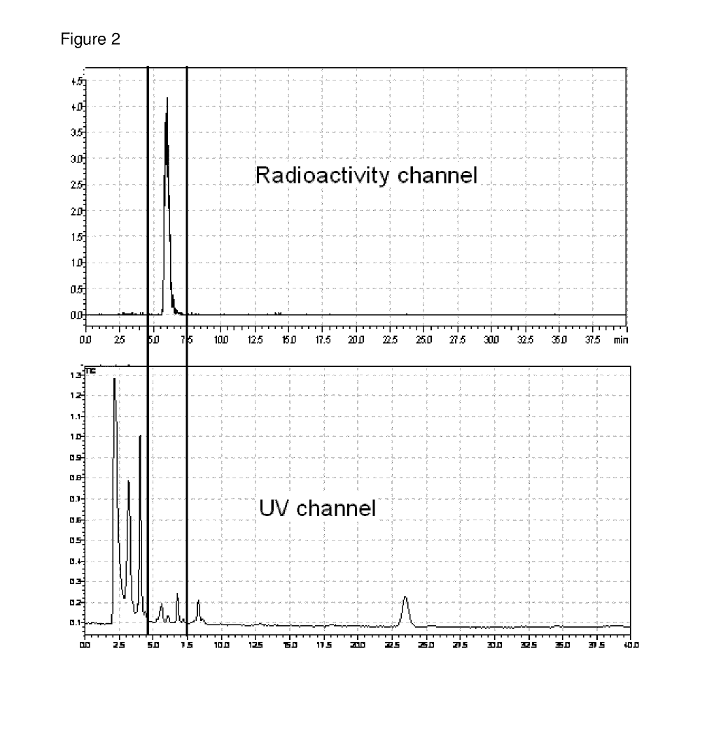

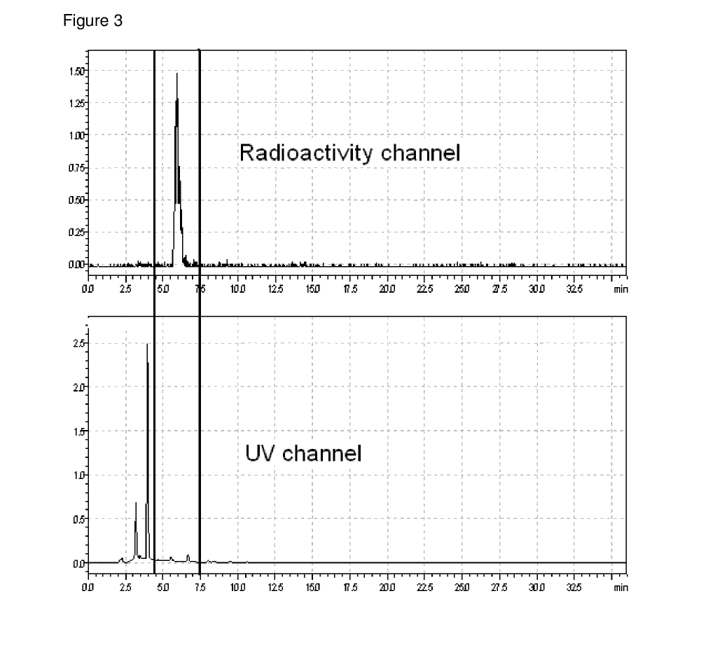

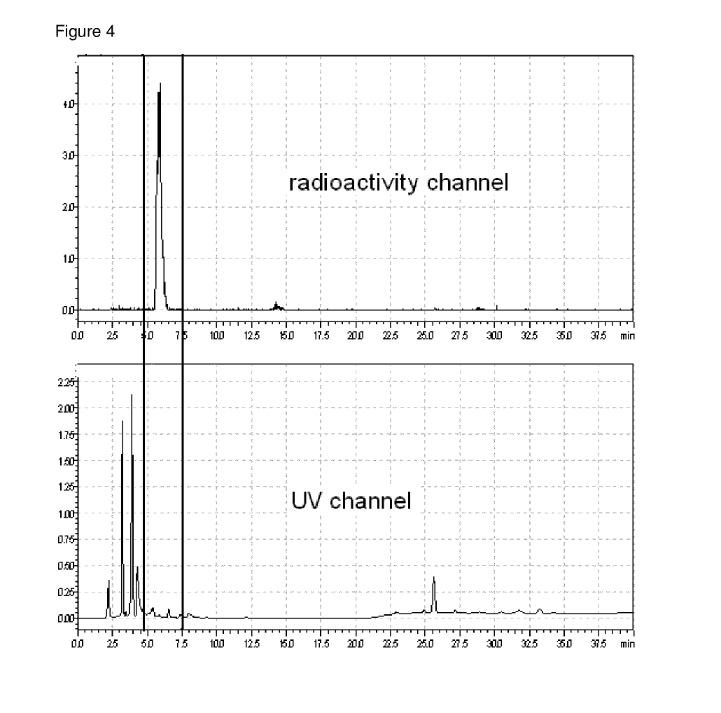

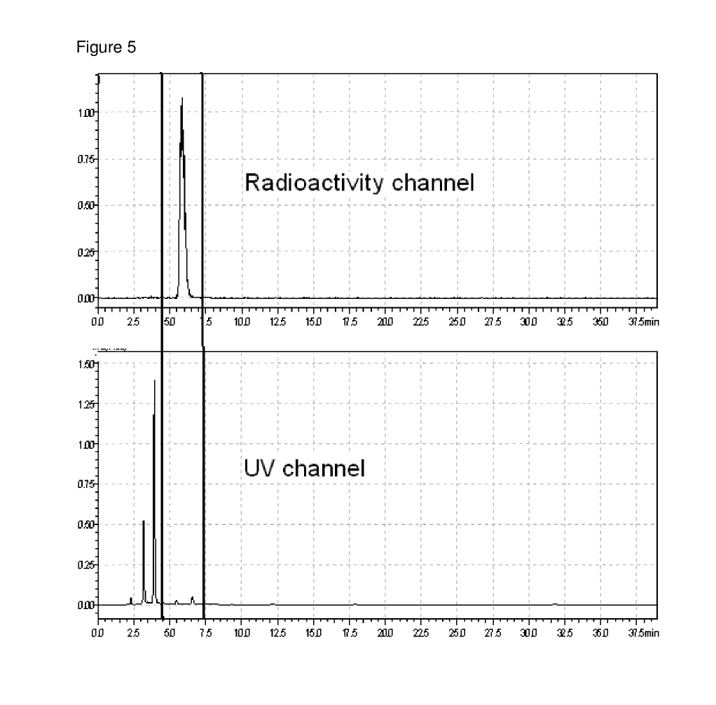

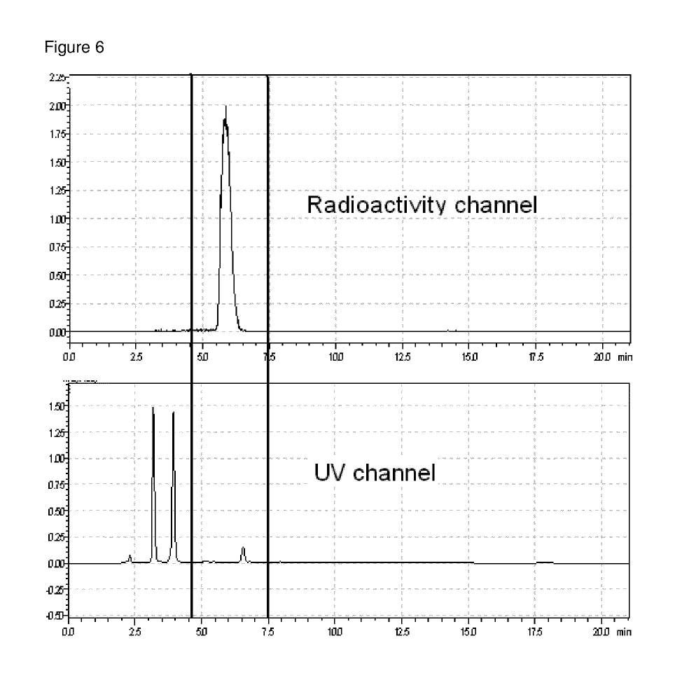

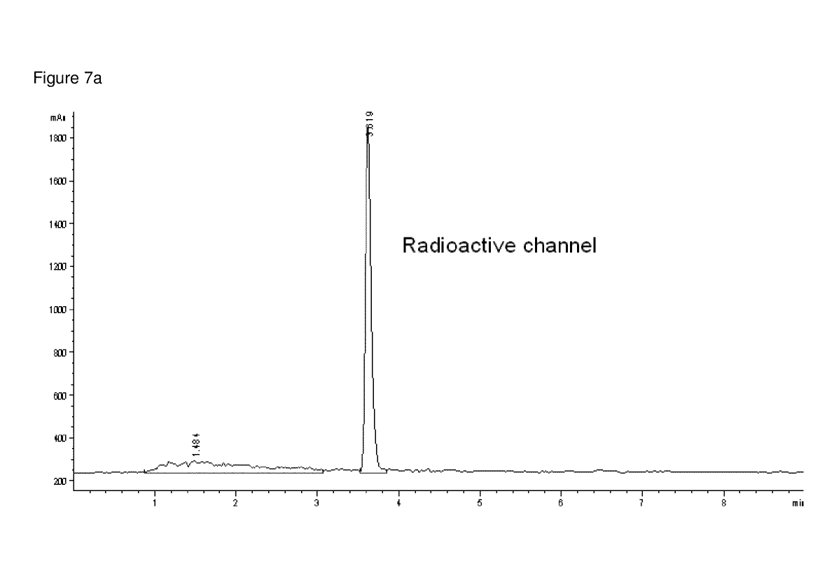

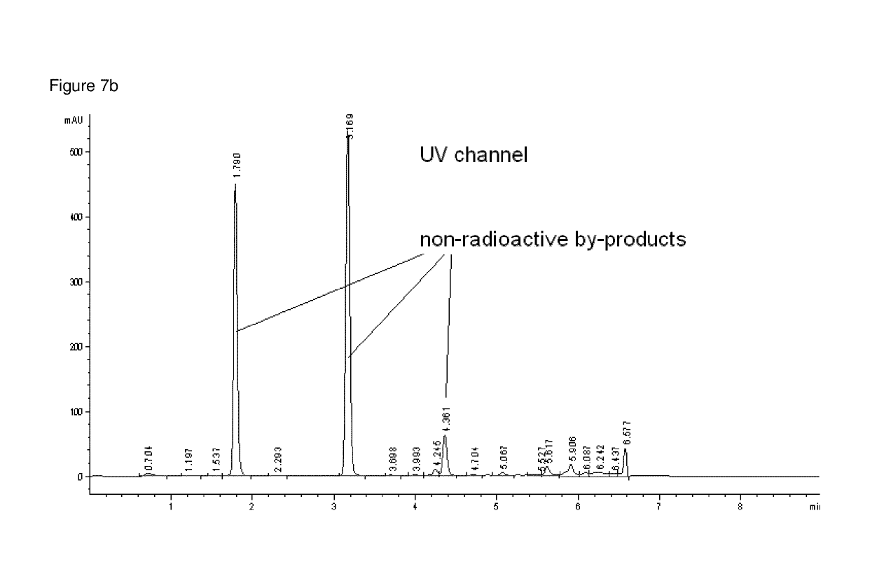

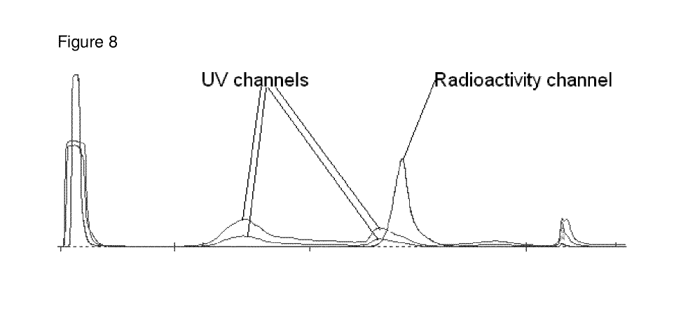

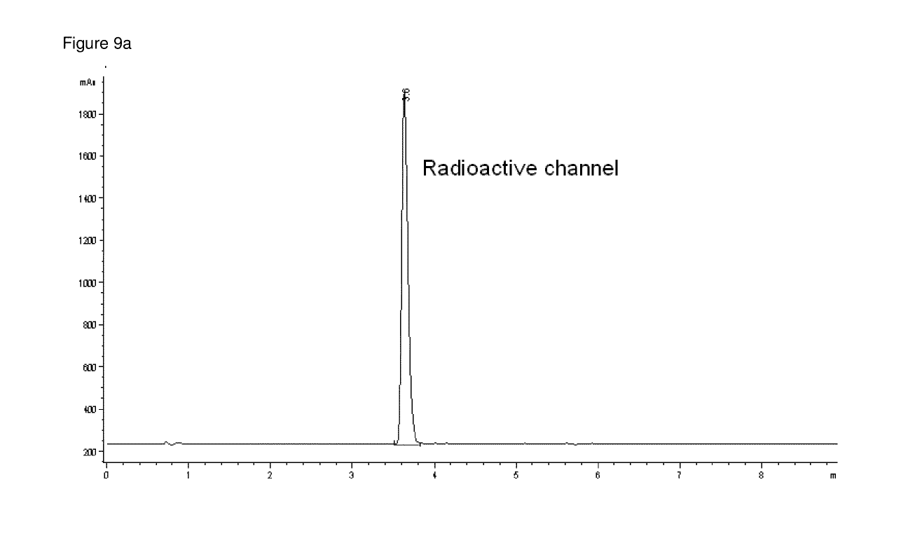

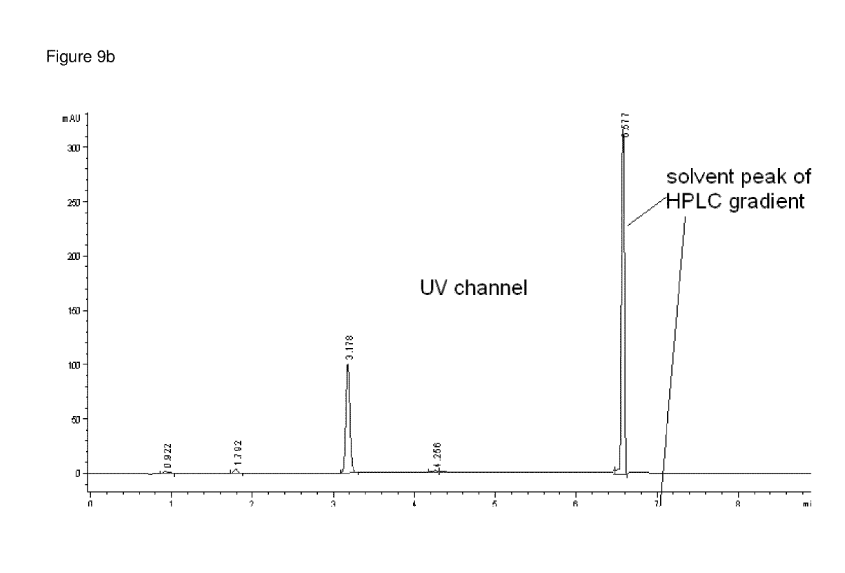

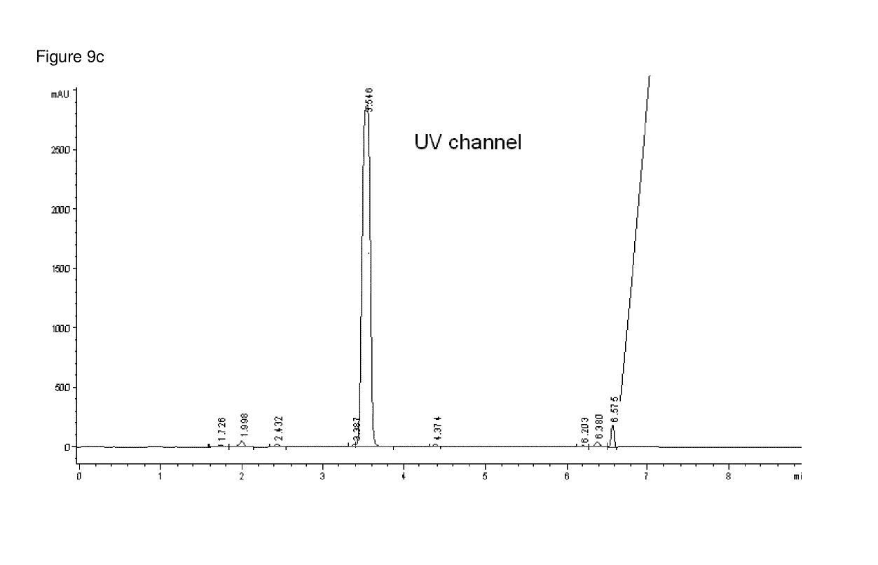

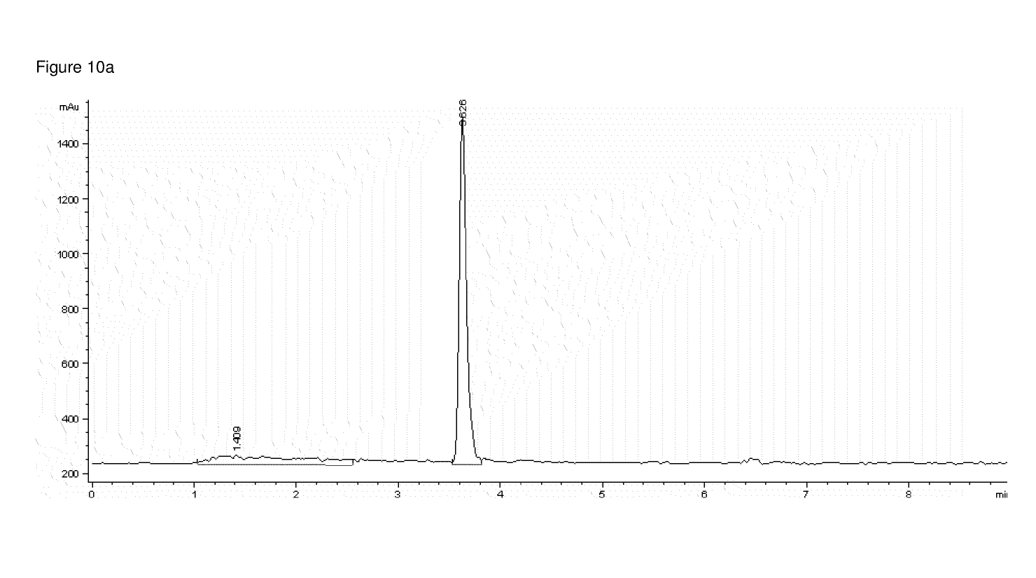

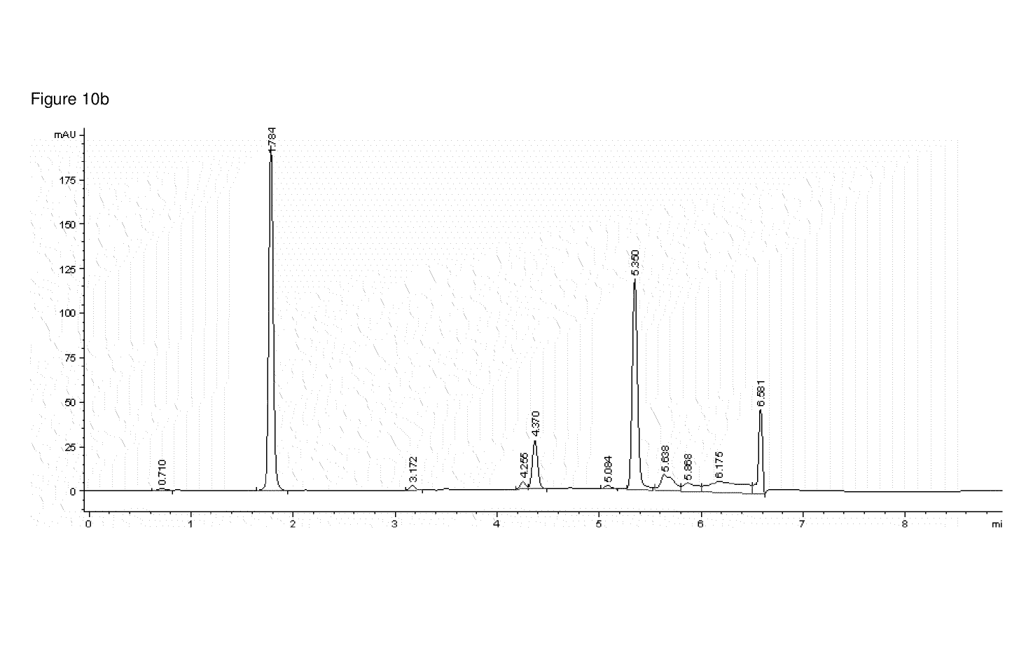

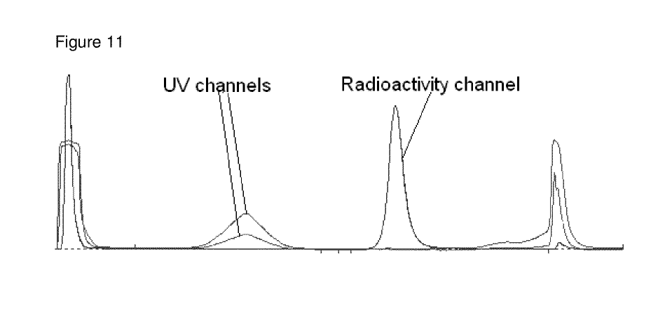

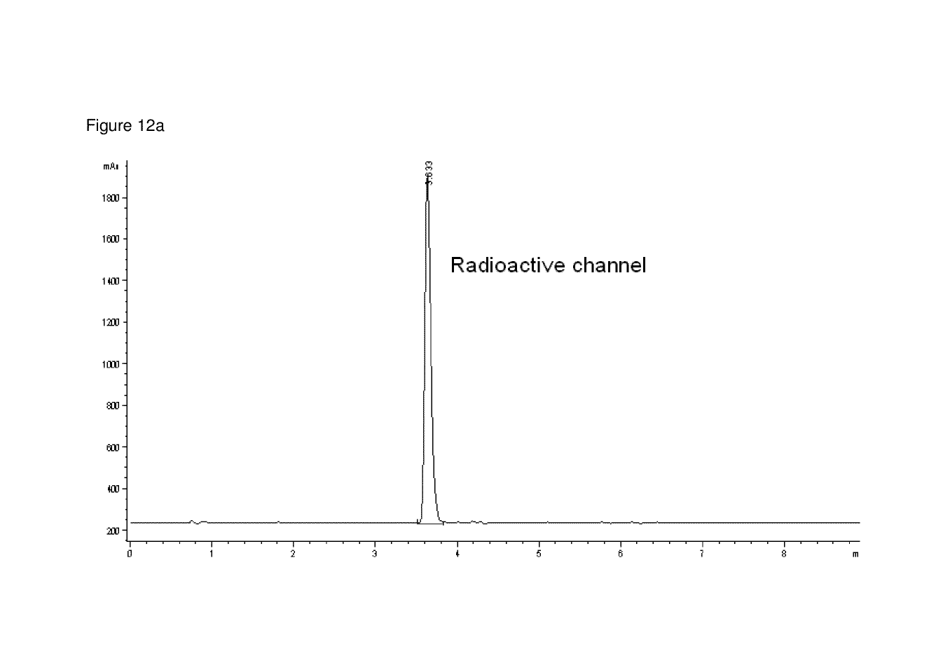

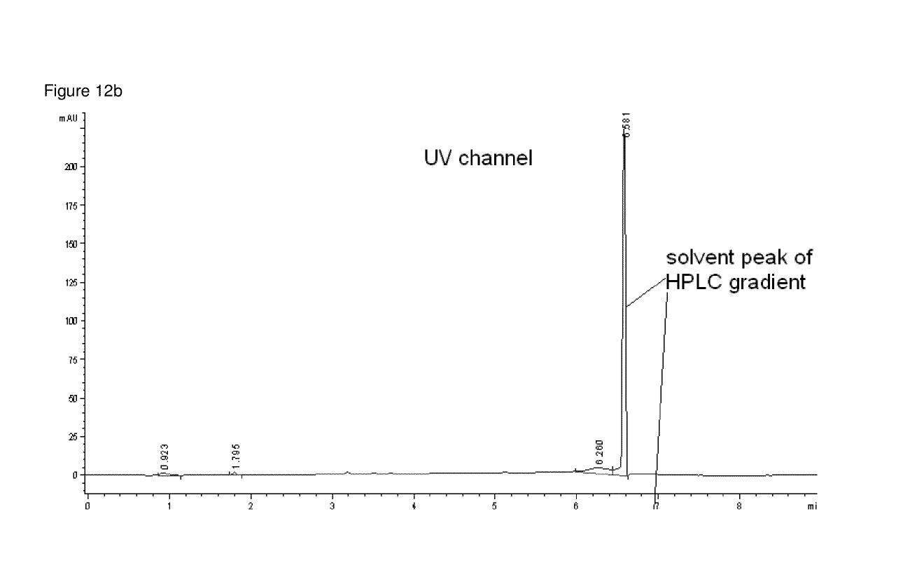

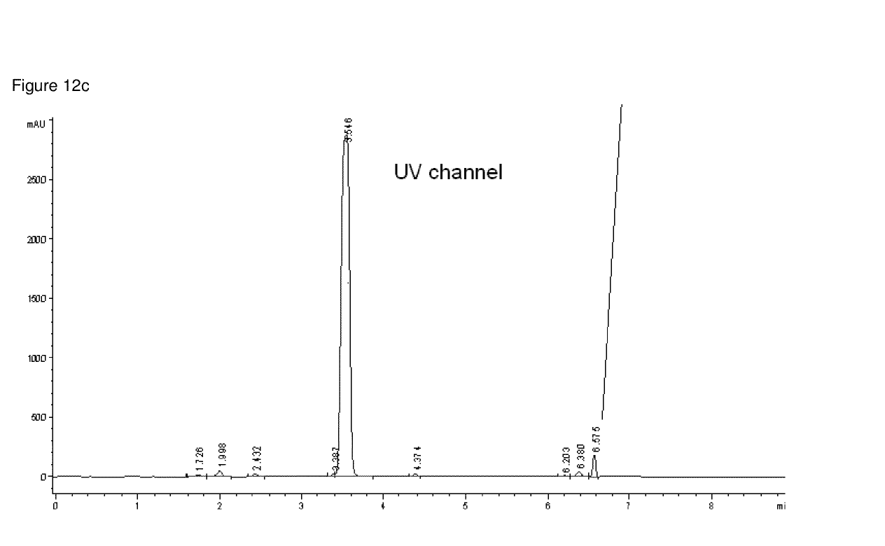

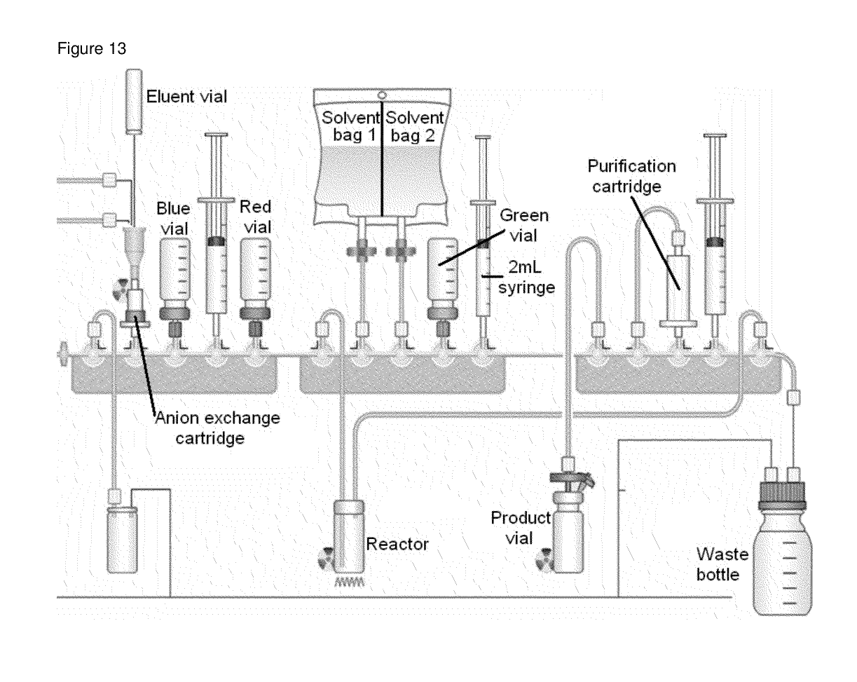

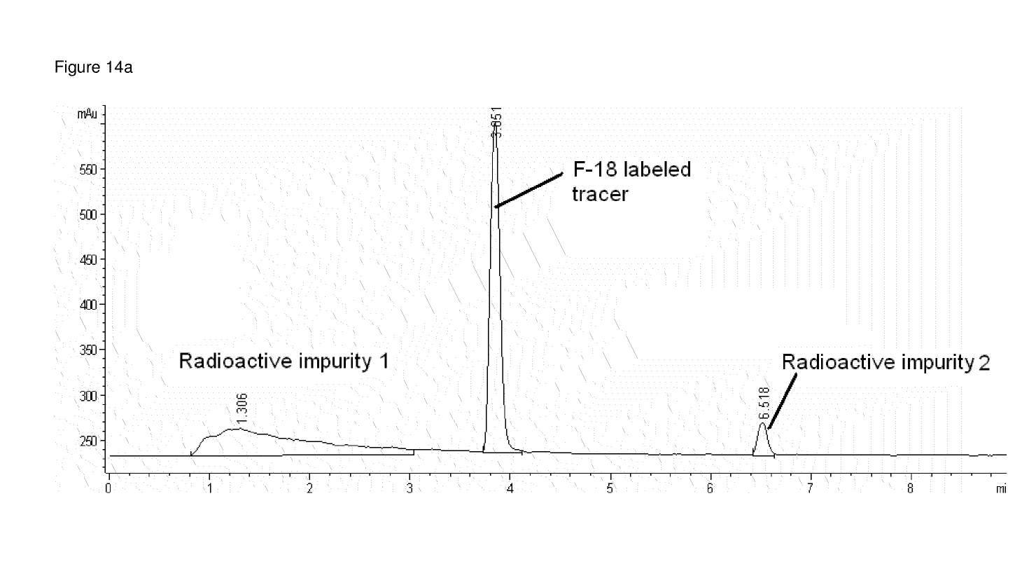

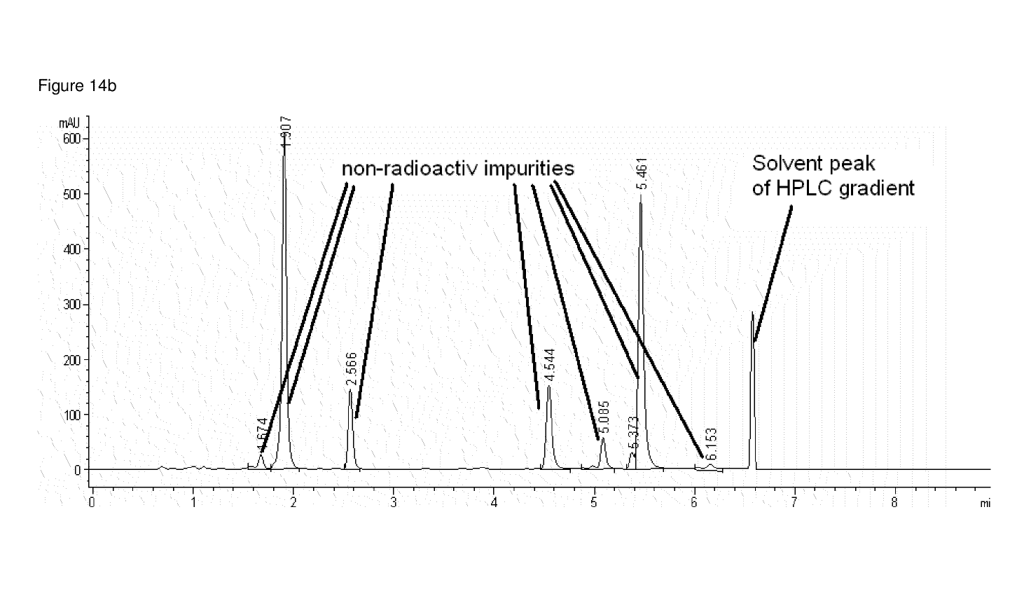

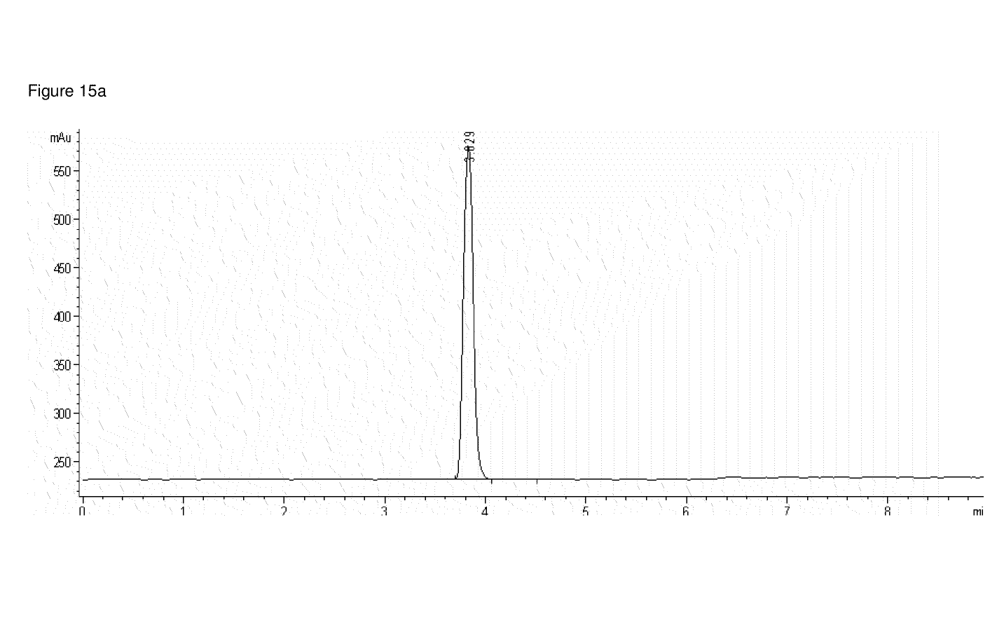

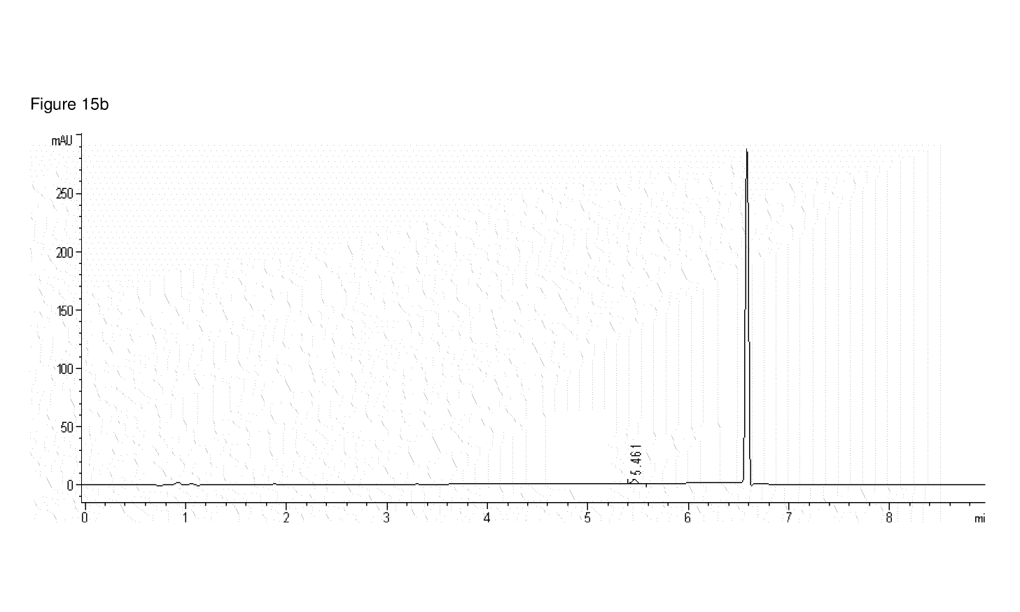

1. A compound of Formula II 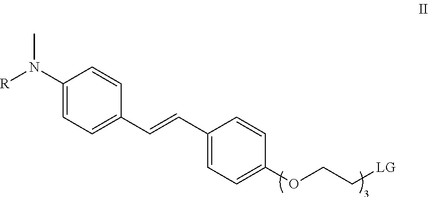
wherein:
R is selected from the group consisting of
a) H,
b) PG,
PG is an “amine-protecting group”,
and
LG is an “Arylsulfonyloxy”.

2. A compound according to claim 1,
Wherein PG is selected from the group consisting of:
a) Boc,
b) Trityl and
c) 4-Methoxytrityl

3. A compound according claim 1,
wherein
Arylsulfonyloxy is selected from the group consisting of
p-Toluenesulfonyloxy, 4-Cyanophenylsulfonyloxy, 4-Bromophenylsulfonyloxy, 4-Nitrophenylsulfonyloxy, 2-Nitrophenylsulfonyloxy, 4-Isopropyl-phenylsulfonyloxy, 2,4,6-Triisopropyl-phenylsulfonyloxy, 2,4,6-Trimethylphenylsulfonyloxy, 4-tert-Butyl-phenylsulfonyloxy, 4-Adamantylphenylsulfonyloxy and 4-Methoxyphenylsulfonyloxy.

4. A compound selected from the group of compounds consisting of
2-[2-(2-{4-[(E)-2-{4-[(tert-butoxycarbonyl)(methyl)amino]phenyl}vinyl]phenoxy}-ethoxy)ethoxy]ethyl-4-bromobenzenesulfonate 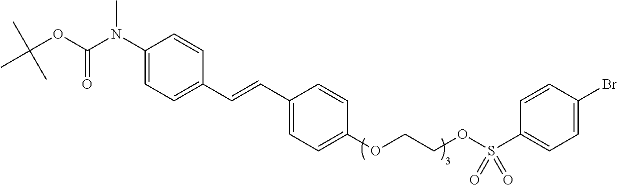
2-[2-(2-{4-[(E)-2-{4-[(tert-butoxycarbonyl)(methyl)amino]phenyl}vinyl]phenoxy}-ethoxy)ethoxy]ethyl-4-(adamantan-1-yl)benzenesulfonate 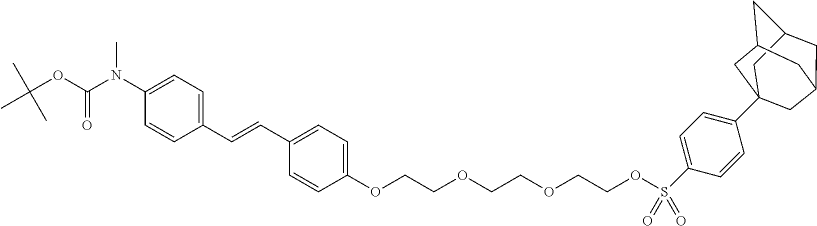 [SMILES: CN(C(=O)OC(C)(C)C)c1ccc(/C=C/c2ccc(OCCOCCOCCOS(=O)(=O)c3ccc(C45CC6CC(CC(C6)C4)C5)cc3)cc2)cc1]
2-[2-(2-{4-[(E)-2-{4-[(tert-butoxycarbonyl)(methyl)amino]phenyl}vinyl]phenoxy}-ethoxy)ethoxy]ethyl-4-cyanobenzenesulfonate 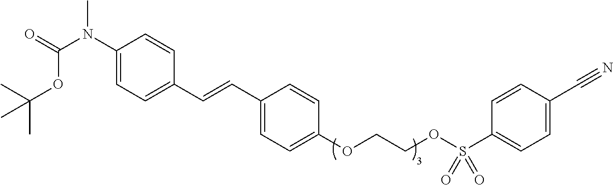
2-[2-(2-{4-[(E)-2-{4-[(tert-butoxycarbonyl)(methyl)amino]phenyl}vinyl]phenoxy}-ethoxy)ethoxy]ethyl-2-nitrobenzenesulfonate 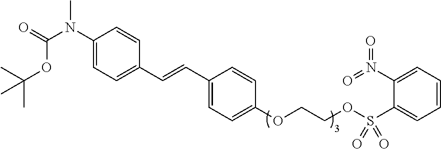
2-[2-(2-{4-[(E)-2-{4-[(tert-butoxycarbonyl)(methyl)amino]phenyl}vinyl]phenoxy}-ethoxy)ethoxy]ethyl-4-methylbenzenesulfonate

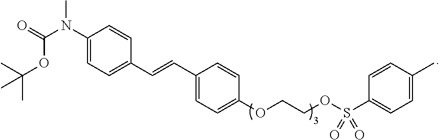

5. A method for producing compound of Formula I by reacting compound of Formula II 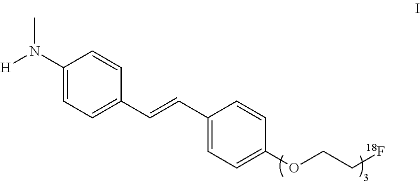
comprising the steps of:
Step 1: Radiolabeling of compound of Formula II with a F-18 fluorinating agent, to obtain compound of Formula I, if R═H or to obtain compound of Formula III, if R=PG 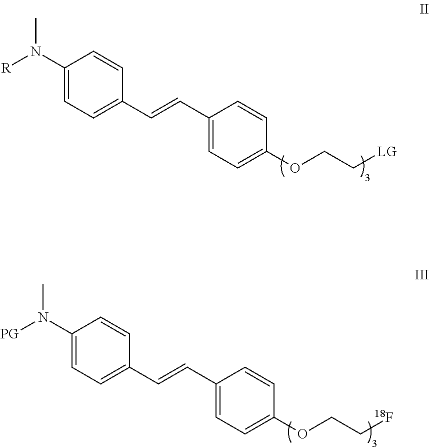
Step 2: If R=PG, cleavage of the protecting group PG to obtain compound of Formula I
Step 3: Purification and Formulation of compound of Formula I
wherein compound of Formula II is as described in claim 1.

6. A method according to claim 5,
wherein in step 1 a compound selected from the following group of compounds
2-[2-(2-{4-[(E)-2-{4-[(tert-butoxycarbonyl)(methyl)amino]phenyl}vinyl]phenoxy}-ethoxy)ethoxy]ethyl-4-bromobenzenesulfonate 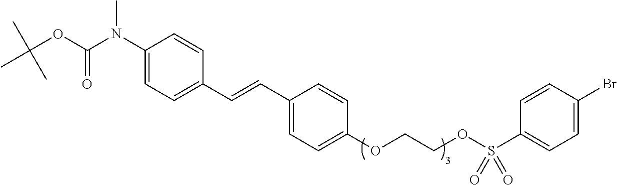
2-[2-(2-{4-[(E)-2-{4-[(tert-butoxycarbonyl)(methyl)amino]phenyl}vinyl]phenoxy}-ethoxy)ethoxy]ethyl-4-(adamantan-1-yl)benzenesulfonate 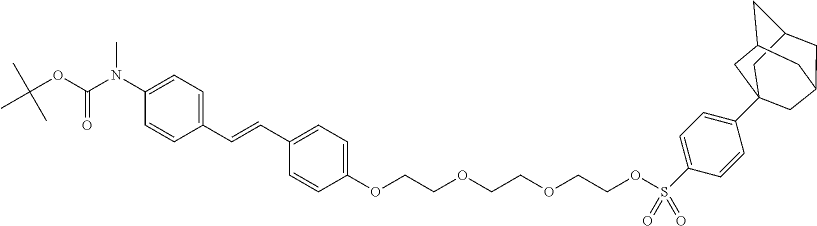 [SMILES: CN(C(=O)OC(C)(C)C)c1ccc(/C=C/c2ccc(OCCOCCOCCOS(=O)(=O)c3ccc(C45CC6CC(CC(C6)C4)C5)cc3)cc2)cc1]
2-[2-(2-{4-[(E)-2-{4-[(tert-butoxycarbonyl)(methyl)amino]phenyl}vinyl]phenoxy}-ethoxy)ethoxy]ethyl-4-cyanobenzenesulfonate 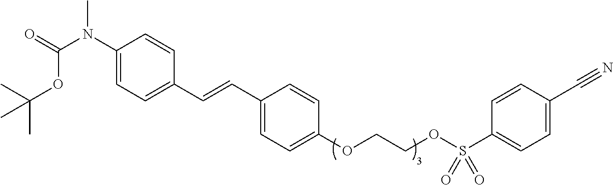
2-[2-(2-{4-[(E)-2-{4-[(tert-butoxycarbonyl)(methyl)amino]phenyl}vinyl]phenoxy}-ethoxy)ethoxy]ethyl-2-nitrobenzenesulfonate 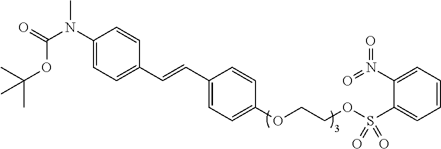
2-[2-(2-{4-[(E)-2-{4-[(tert-butoxycarbonyl)(methyl)amino]phenyl}vinyl]phenoxy}-ethoxy)ethoxy]ethyl-4-methylbenzenesulfonate 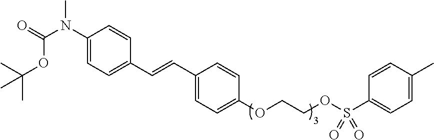
is used.

7. A method according to claim 5, wherein the method is a fully automated method.

8. A kit, comprising at least one sealed container, containing a compound of formula II, 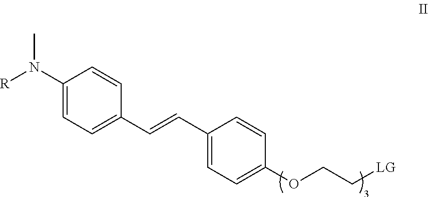
Wherein
R is selected from the group comprising
a) H,
b) PG,
PG is an “amine-protecting group”,
and
LG is Arylsulfonyloxy.

9. A kit according to claim 8,
wherein
PG is selected from the group comprising:
a) Boc,
b) Trityl and
c) 4-Methoxytrityl

10. A kit according to claim 8,
Wherein
Arylsulfonyloxy is selected from the group comprising:
a) p-Toluenesulfonyloxy
b) (2-Nitrophenyl)sulfonyloxy,
c) (4-Cyanophenyl)sulfonyloxy
d) (4-Bromophenyl)sulfonyloxy,
e) (4-Adamantylphenyl)sulfonyloxy.

11. A kit comprising at least one sealed container containing a compound as defined by claim 4.
